(19) United States
(12) Patent Application Publication
(10) Pub. No.: US 2025/0236868 A1
(43) Pub. Date: Jul. 24, 2025

(54) COMPOSITIONS AND METHODS FOR TREATING LIVER DISEASES WITH SIRNAS TARGETING CIDEB

(71) Applicant: The Board of Regents of The University of Texas System, Austin, TX (US)

(72) Inventors: Hao Zhu, Dallas, TX (US); Zixi Wang, Dallas, TX (US); Lin Li, Dallas, TX (US)

(21) Appl. No.: 18/853,893

(22) PCT Filed: Apr. 7, 2023
(86) PCT No.: PCT/US2023/065557

§ 371 Date: Oct. 3, 2024

Related U.S. Application Data
(60) Provisional application No. 63/328,557, filed on Apr. 7, 2022.

(51) Int. Cl. C12N 15/113; C12N 9/22

(52) U.S. Cl. CPC C12N 15/113; C12N 9/22; C12N 2310/14; C12N 2310/20; C12N 2310/321; C12N 2310/351

(57) ABSTRACT

Disclosed herein are compositions comprising siRNAs capable of downregulating Cell Death-Inducing DFF45-like Effector Protein B (CIDEB) gene expression or a variant thereof. Also disclosed herein are methods of using such compositions in the treatment of a liver disease or injury, such as fatty liver disease (FLD), non-alcoholic fatty liver disease (NAFLD) or non-alcoholic steatohepatitis (NASH).

### CROSS REFERENCE TO RELATED APPLICATIONS

This application claims the benefit of the U.S. Provisional Application No. 63/328,557, filed Apr. 7, 2022, the disclosures of which are incorporated herein by reference in their entireties for all purposes.

### SEQUENCE LISTING

This application contains a Sequence Listing that has been submitted in xml format via EFS-Web and is hereby incorporated by reference in its entirety. The xml copy is named 106546-755471.xml and is 87 KB in size.

### BACKGROUND

### 1. Field

The present inventive concept is directed to compositions and methods of use thereof for liver disease treatment, specifically to metabolic liver diseases, including but not limited to non-alcoholic fatty liver disease (NAFLD) and nonalcoholic steatohepatitis (NASH).

### 2. Discussion of Related Art

NAFLD is a spectrum of chronic liver disorders, which encompass, among others nonalcoholic fatty liver (NAFL) and nonalcoholic steatohepatitis (NASH). NASH, a common cause of chronic liver disease, is defined as having at least 5% hepatic steatosis and inflammation with or without fibrosis. Over time and without treatment, NASH may progress to cirrhosis and even hepatocellular carcinoma (HCC). Moreover, NAFLD/NASH is strongly associated with obesity and type II diabetes which together affect over 50% of the US population, leading to a heavy economic burden. Unfortunately, therapeutic options for NASH remain limited, with only slight benefits observed from vitamin E or obeticholic acid treatment. While NASH is the leading cause of chronic liver disease and cirrhosis, there are currently no clinically approved therapies. As such, new targets, therapeutics, and combinations thereof are needed to accelerate clinical progress in the treatment of liver diseases.

### SUMMARY OF THE INVENTION

The present disclosure is based, in part, on the finding that positive selection of somatic mutations in NASH patient livers can increase cell fitness and competitiveness through decreased lipid accumulation in NASH. Somatic mutation sequencing in human cirrhosis patients identified genetic alterations that promote clonal fitness, likely through the reversal of lipotoxicity. These efforts identified CIDEB (Cell Death-Inducing DFF45-like Effector Protein B). Accordingly, the present disclosure provides for novel compositions for modulating CIDEB expression and methods of preventing, attenuating and/or treating liver diseases.

In some aspects, the current disclosure encompasses a composition comprising a nucleic acid that downregulates expression of Cell Death-Inducing DFF45-like Effector Protein B (CIDEB) or a variant thereof. In some aspects, the nucleic acid that downregulates expression of CIBED comprises a siRNA, a cluster regularly interspaced short palindromic repeats (CRISPR) related nucleic acid, a single guide RNA (sgRNA), a CRISPR-RNA (crRNA), or a trans-activating crRNA (tracrRNA). In some aspects, the nucleic acid that downregulates gene expression of CIDEB or a variant thereof is a small interfering RNA (siRNA) molecule. In some aspects, the plasmid or the viral vector comprises a nucleic acid encoding the siRNA molecule as disclosed herein. In some aspects, the siRNA molecule comprises a nucleotide sequence that is 2 to 30 nucleotides in length and is at least 80% homologous to at least 2 to 30 contiguous nucleotides of a human CIDEB cDNA sequence, wherein the human CIDEB cDNA sequence is SEQ ID NO: 1. In some aspects, the siRNA molecule targets the open reading frame or the 5′ or 3′ UTRs of the CIDEB gene. In some aspects, the siRNA molecule comprises at least one sense sequence, at least one antisense sequence, or at least one sense sequence and at least one antisense sequence. In some aspects, the siRNA molecule comprises a nucleotide sequence SEQ ID NOs: 2-97 or any combination thereof. In some aspects, the at least one sense sequence comprises SEQ ID NOs: 2-49. In some aspects, the at least one antisense sequence comprises SEQ ID NOs: 50-95.

In some aspects, the current disclosure also encompasses a composition comprising a nucleic acid molecule that downregulates expression of CIBED, wherein the nucleic acid is a sgRNA or encodes an sgRNA. In some aspects, the composition comprises a plasmid or a viral vector, wherein the plasmid or the viral vector comprises a first nucleic acid encoding the sgRNA molecule as disclosed herein and optionally a second nucleic acid encoding an RNA guided nuclease. In some aspects, the RNA guided nuclease is a Cas endonuclease.

In some aspects, the siRNA molecule as disclosed herein specifically downregulates gene expression of at least one variant of CIBED. In some aspects, the sgRNA molecule specifically downregulates gene expression of at least one variant of CIBED. In some aspects, the at least one variant of CIDEB is associated with a liver disease. Non-limiting examples of liver disease comprises fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer, or any combination thereof.

In some aspects, the nucleic acid molecule as disclosed herein may be conjugated to least one targeting ligand. In some aspects, at least one targeting ligand comprises a liver targeting ligand. In some aspects, the liver targeting ligand comprises at least one N-acetylgalactosamine (GalNAc) conjugate. In some aspects, the nucleic acid molecule is conjugated to about one to about three GalNAc conjugates. In some aspects, the nucleic acid molecule comprises at least one chemical modification. In some aspects, the nucleic acid molecule comprises a modification at least one ribosugar moiety of its nucleotide sequence. In some aspects, the at least one ribosugar moiety is modified with 22′-O-methyl (2′OMe), 2′-deoxy-2′-fluoro (2′F), 2′-deoxy, 5-C-methyl, 2′-O-(2-methoxyethyl) (MOE), 4′-thio, 2′-amino, 2′-C-allyl, or any combination thereof. In some aspects, less than about 10% to about 70% of ribosugar moieties of the total nucleotide sequence is modified.

In some aspects, the current disclosure also encompasses a pharmaceutical composition comprising any one of the compositions as disclosed herein and at least one pharmaceutically acceptable carrier. In some aspects, the pharmaceutical composition further comprises a nanoparticle. In some aspects, the pharmaceutical composition further comprises a lipid.

In some aspects, the current disclosure also encompasses a method of treating a subject in need thereof, the method comprising administrating a therapeutically effective amount of the composition as disclosed herein, or the pharmaceutical composition as disclosed herein. In some aspects, the subject in need thereof, is a human subject having or suspected of having a liver disease. In some aspects, the liver disease comprises fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer, or any combination thereof. In some aspects, the method of administering comprises parenteral administration. In some aspects, the administration of a therapeutically effective amount of the composition as disclosed here or the pharmaceutical composition as disclosed herein increases life expectancy of the subject compared to an untreated subject with identical disease condition and predicted outcome. In some aspects of the method, administration of a therapeutically effective amount of the composition as disclosed herein or the pharmaceutical composition as disclosed herein increases liver function of the subject compared to an untreated subject with identical disease condition and predicted outcome. In some aspects of the method, administration of a therapeutically effective amount of the composition as disclosed herein or the pharmaceutical composition as disclosed herein attenuates liver fibrosis in the subject compared to an untreated subject with identical disease condition and predicted outcome. In some aspects, the administration of a therapeutically effective amount of the composition as disclosed herein or the pharmaceutical composition as disclosed herein prevents additional liver fibrosis in the subject compared to an untreated subject with identical disease condition and predicted outcome.

In some aspects, the current disclosure also encompasses a kit comprising: a container holding the composition as disclosed here or the pharmaceutical composition as disclosed herein, a pharmaceutical administrative means; and instructions for use.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1A show a screen of 63 known NASH related genes. The scheme for the experiment is shown.

FIG. 1B shows the results of the screen of 63 known NASH related genes. The clones that expanded the most were often associated with deletion of lipogenesis genes.

FIG. 1C provides a with the top gene hits from the screen.

FIG. 1D shows the pathways in which the gene hits are involved. This screen served to show that genes mutated in human cirrhosis tissues are likely to be good drug targets for NASH. CIBED is one such gene that is frequently mutated in human liver disease.

FIG. 2A shows a luciferase-based screen used to test the relative knockdown efficacy for various CIDEB siRNAs.

FIG. 2B shows CIDEB protein levels that were examined using western blot analysis after siRNA knockdown.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

With increasing over-nutrition and obesity, non-alcoholic fatty liver disease (NAFLD) is rapidly becoming the leading cause of liver disease in the world. NAFLD is usually conceptualized at the organismal and tissue levels; however, little thought has been given to genetic heterogeneity within clones of the liver. Somatic mutations are common in most healthy individuals, and there is accumulating evidence that mutation burden increases with age and chronic tissue damage. The present disclosure is based, in part, on the novel finding that positive selection of somatic mutations in NASH patient livers can increase cell fitness and competitiveness through decreased lipid accumulation in NASH. Genomic sequencing of chronic liver disease samples identified genes that when mutated, promote clonal fitness likely through the reversal of lipotoxicity, including CIDEB (Cell Death-Inducing DFF45-like Effector Protein B). Accordingly, provided herein are compositions and methods of use thereof for liver disease treatment, specifically to metabolic liver diseases, including but not limited to non-alcoholic fatty liver disease (NAFLD) and nonalcoholic steatohepatitis (NASH).

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

As used herein, the term “about,” can mean relative to the recited value, e.g., amount, dose, temperature, time, percentage, etc., +10%, +9%, +8%, 7%, +6%, +5%, +4%, +3%, +2%, or #1%.

The terms “comprising,” “including,” “encompassing” and “having” are used interchangeably in this disclosure. The terms “comprising,” “including,” “encompassing” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

“Biomarker” as used herein refers to any biological molecules (e.g., nucleic acids, genes, peptides, proteins, lipids, hormones, metabolites, and the like) that, singularly or collectively, reflect the current or predict future state of a biological system. Thus, as used herein, the presence or concentration of one or more biomarkers can be detected and correlated with a known condition, such as a disease state. In some aspects, detecting the presence and/or concentration of one or more biomarkers herein may be an indication of a liver disease risk in a subject. In some other aspects, detecting the presence and/or concentration of one or more biomarkers herein may be used in treating and/or preventing a chronic liver disease in a subject.

As used herein, the terms “treat”, “treating”, “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder.

The term “biomolecule” as used herein refers to, but is not limited to, proteins, enzymes, antibodies, DNA, siRNA, and small molecules. “Small molecules” as used herein can refer to chemicals, compounds, drugs, and the like.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

It should also be understood that, unless clearly indicated to the contrary, in any methods claimed herein that include more than one step or act, the order of the steps or acts of the method is not necessarily limited to the order in which the steps or acts of the method are recited.

### II. Compositions

### (a) CIDEB

In certain embodiments, compositions for use in the methods disclosed herein can modulate CIDEB (Cell Death-Inducing DFF45-like Effector Protein B) gene. The CIDEB gene encodes CIDEB protein, which is the major CIDE-family member that is active in hepatocytes. CIDEB promotes lipid storage under normal diet conditions and promotes the formation of triacylglyceride-enriched VLDL particles in hepatocytes.

As used herein, compositions “modulating” CIDEB can include any biomolecule(s) capable of decreasing CIDEB gene expression, decreasing CIDEB protein expression, decreasing CIDEB activity, or a combination thereof. In some aspects, biomolecule(s) herein capable of modulating CIDEB can be an inhibitor of CIDEB. As used herein, an inhibitor of CIDEB can inhibit CIDEB direct activity, inhibit CIDEB indirect activity, decrease expression of the CIDEB gene, decrease expression of the CIDEB protein, or a combination thereof.

In certain embodiments, compositions for use in the methods disclosed herein can include a nucleic acid molecule. The term “nucleic acid molecule” as used herein refers to a molecule having nucleotides. The nucleic acid can be single, double, or multiple stranded and may comprise modified or unmodified nucleotides or non-nucleotides or various mixtures and combinations thereof. In some embodiments, a nucleic acid molecule for use herein can be a double-stranded RNA. In some examples, a double stranded RNA suitable for use herein can be small temporal RNA, small nuclear RNA, small nucleolar RNA, short hairpin RNA, microRNA, or the like. In certain embodiments, a double stranded RNA suitable for use herein can be a small interfering RNA (siRNA).

The term “siRNA” as used herein refers to small inhibitory RNA duplexes that induce the RNA interference (RNAi) pathway. A siRNA molecule disclosed herein may be capable of silencing, reducing, and/or inhibiting expression of a target gene (e.g., CIDEB). These molecules can vary in length (generally about 5-50 base pairs) and contain varying degrees of complementarity to their target mRNA in the antisense strand. Some, but not all, siRNA have unpaired overhanging bases on the 5′ or 3′ end of the sense strand and/or the antisense strand. The term “siRNA” as used herein can includes duplexes of two separate strands, as well as single strands that can form hairpin structures comprising a duplex region.

In certain embodiments, siRNA molecules disclosed herein may be any interfering RNA with a duplex length of about 2-60, about 5-50, or about 10-40 nucleotides in length, more typically about 2-30, about 5-25, or about 10-25 nucleotides in length. In some embodiments, SIRNA molecule disclosed herein may have a nucleotide sequence that is about 2 to about 30 nucleotides in length, about 5 to about 25 nucleotides in length, or about 10 to about 20 nucleotides in length. In some embodiments, siRNA molecule disclosed herein may have a nucleotide sequence that is about 10 nucleotides in length, about 11 nucleotides in length, about 12 nucleotides in length, about 13 nucleotides in length, about 14 nucleotides in length, about 15 nucleotides in length, about 14 nucleotides in length, about 15 nucleotides in length, about 16 nucleotides in length, about 17 nucleotides in length, about 18 nucleotides in length, about 19 nucleotides in length, about 20 nucleotides in length, about 21 nucleotides in length, about 22 nucleotides in length, about 23 nucleotides in length, about 24 nucleotides in length, or about 25 nucleotides in length. Each complementary sequence of the double-stranded siRNA may be about 2-60, about 5-50, about 10-40, about 2-30, about 5-25, or about 10-25 nucleotides in length, but other noncomplementary sequences may be present. For example, siRNA duplexes may comprise 3′ overhangs of about 1 to about 4 or more nucleotides and/or 5′ phosphate termini comprising about 1 to about 4 or more nucleotides.

In certain embodiments, siRNA molecules disclosed herein may have about 2-60, about 2-50, about 2-40, or about 2-30 contiguous nucleotides of homology with a target (e.g., CIDEB) nucleotide sequence. In some aspects, a target nucleotide sequence herein may be a human CIDEB nucleotide sequence or a variant thereof. In some other some aspects, a target nucleotide sequence herein may be human CIDEB, RefSeq: NM_001393339.1 or a variant thereof. In still some other aspects, a target nucleotide sequence herein may be SEQ ID NO: 1 as follows, or a variant thereof:

Human CIDEB (SEQ ID NO: 1):
CCCTTCCGGTGGAGCCAGCGCTGCGACCGCCTGCAGAAGGTTGACTGCG
TGGTAGGGGGCCCAGAGCAAGCCGAAGGCAAGCACGATGGCGCTCACCA
GCCGGCCCACCCGCGCCCCGTGCCGCCCGGAGCCCCAGCGGGCGCCCCG
CAGCCGTGCCAGCGTCACGCTGTAGCAGCCGAGCATCAGCCCGAAAGGA
AGCACGAAAGCGGTCAGAGTCTCCAGGCTCAGGTGGGCGGCGGCGTGGA
CCGGCGACGGGTGGCACAGCTGGCATACGCGGTCCCTCCACAGGTGGCG
GTAGACGGCGGCCGGGACGGCGAGCAACAGGGCGGCCAGCCAGACCGCC
AGCAGCAGGCGGCGGGCCAGGGCCGGGCTGCGCAGCCGAGGCGCCAGGA
AGGGGCGGGTGACTGCGAGGCAGCGCTGCAGGCTGAGCAGGCCGGTGAG
CAGCACGCTGGCGTACATGCTGAGCGCGCACACGTAGTACACCGCCTTG
CAGCCCGCCTGGCCCAGCGGCCAGGCCTGCCGGGTCAGGAAGGCCACAA
AGAGCGGCGTGAGCAGCAGCACCGCGCCGTCGGCCAGCGCCAGGTGCAG
CACAAGCGTGGCCGCCAGCGGTCGCCCCCGTGCAGGCCGCCAGCCCGCC
AAGCTCCACACCACGAAGCCGTTGCCAGGCAGCCCCAGCAGCGCCGCCA
GCAGCAGGAAGGCTGTGCCTGTGGCCCGCGAAGTCTTCCAGCTCAGCAG
TGTCTCGTTCCCTGGGGGACGGTAGCAGACCGACATCCTTCTGGGCCTA
CAGTGAGAGTCTCAGCTTCCATGCAACTGTCCATCACGGCTGCAACTGA
AATCAGAGCTGGGACACAGCGCACCAGAAGCTAAAGTCTTGATGCCATC
AAAGGACATCCCTGCCCCATTCACATCTCTGTCACGTCCACTAATCGGC
AAAAGGAGAAAAGTGAGAGAAGATGACCTAAGTGTGACTGCAGCAGGCA
GCTCTGGAAAATGAAGCCAGAGCAGTGAGCCAGCCCCTCCTCCGACCAA
GGAGGAAGGAAAGAGCAGCCCCAGCACAGGAGAGAACCACCCAGCCCAG
AAGTTCCAGGGAAGGAACTCTCCGGTCCACCATGGAGTACCTCTCAGCT
CTGAACCCCAGTGACTTACTCAGGTCAGTATCTAATATAAGCTCGGAGT
TTGGACGGAGGGTCTGGACCTCAGCTCCACCACCCCAGCGACCTTTCCG
TGTCTGTGATCACAAGCGGACCATCCGGAAAGGCCTGACAGCTGCCACC
CGCCAGGAGCTGCTAGCCAAAGCATTGGAGACCCTACTGCTGAATGGAG
TGCTAACCCTGGTGCTAGAGGAGGATGGAACTGCAGTGGACAGTGAGGA
CTTCTTCCAGCTGCTGGAGGATGACACGTGCCTGATGGTGTTGCAGTCT
GGTCAGAGCTGGAGCCCTACAAGGAGTGGAGTGCTGTCATATGGCCTGG
GACGGGAGAGGCCCAAGCACAGCAAGGACATCGCCCGATTCACCTTTGA
CGTGTACAAGCAAAACCCTCGAGACCTCTTTGGCAGCCTGAATGTCAAA
GCCACATTCTACGGGCTCTACTCTATGAGTTGTGACTTTCAAGGACTTG
GCCCAAAGAAAGTACTCAGGGAGCTCCTTCGTTGGACCTCCACACTGCT
GCAAGGCCTGGGCCATATGTTGCTGGGAATTTCCTCCACCCTTCGTCAT
GCAGTGGAGGGGGCTGAGCAGTGGCAGCAGAAGGGCCGCCTCCATTCCT
ACTAAGGGGCTCTGAGCTTCTGCCCCCAGAATCATTCCAACCGACCCAC
TGCAAAGACTATGACAGCATCAAATTTCAGGACCTGCAGACAGTACAGG
CTAGATAACCCACCCAATTTCCCCACTGTCCTCTGATCCCCTCGTGACA
GAACCTTTCAGCATAACGCCTCACATCCCAAGTCTATACCCTTACCTGA
AGAATGCTGTTCTTTCCTAGCCACCTTTCTGGCCTCCCACTTGCCCTGA
AAGGCCAAGATCAAGATGTCCCCCAGGCATCTTGATCCCAGCCTGACTG
CTGCTACATCTAATCCCCTACCAATGCCTCCTGTCCCTAAACTCCCCAG
CATACTGATGACAGCCCTCTCTGACTTTACCTTGAGATCTGTCTTCATA
CCCTTCCCCTCAAACTAACAAAAACATTTCCAATAAAAATATCAAATAT
TTACCACTAA

In certain embodiments, siRNA molecules disclosed herein may have a nucleotide sequence that is about 2-30 contiguous in length and is at least about 80% homologous (e.g., about 80%, about 85%, about 90%, about 95%, or about 99%) to at least about 2 to about 30 contiguous nucleotides of a human CIDEB cDNA sequence. In some embodiments, siRNA molecules disclosed herein may have a nucleotide sequence that is about 2-30 contiguous in length and is at least 80% homologous (e.g., about 80%, about 85%, about 90%, about 95%, or about 99%) to at least about 2 to about 30 contiguous nucleotides of the human CIDEB sequence RefSeq: NM_001393339.1 or a variant thereof. In some embodiments, siRNA molecules disclosed herein may have a nucleotide sequence that is about 2-30 contiguous in length and is at least 80% homologous (e.g., about 80%, about 85%, about 90%, about 95%, or about 99%) to at least about 2 to about 30 contiguous nucleotides of SEQ ID NO: 1. In some other embodiments, siRNA molecules disclosed herein may have a nucleotide sequence that is about 2-30 contiguous in length and is homologous to at least about 2 to about 30 contiguous nucleotides of SEQ ID NO: 1.

A siRNA molecule disclosed herein may be synthesized in any of a number of conformations. One skilled in the art would recognize the type of siRNA conformation to be used for a particular purpose. Examples of siRNA conformations include, but need not be limited to, a double-stranded polynucleotide molecule assembled from two separate stranded molecules, wherein one strand is the sense strand and the other is the complementary antisense strand; a double-stranded polynucleotide molecule assembled from a single-stranded molecule, where the sense and antisense regions are linked by a nucleic acid-based or non-nucleic acid-based linker; a double-stranded polynucleotide molecule with a hairpin secondary structure having complementary sense and antisense regions; or a circular single-stranded polynucleotide molecule with two or more loop structures and a stem having self-complementary sense and antisense regions. In the case of the circular polynucleotide, the polynucleotide may be processed either in vivo or in vitro to generate an active double-stranded siRNA molecule.

In certain embodiments, siRNA molecules disclosed herein may be double stranded siRNA molecules. In some aspects, double stranded siRNA molecules disclosed herein may have at least one sense sequence. In some other aspects, double stranded siRNA molecules disclosed herein may have at least one antisense sequence. In still some other aspects, double stranded siRNA molecules disclosed herein may have at least one antisense sequence and at least one sense sequence. In some embodiments, double stranded siRNA molecules disclosed herein may have at least one antisense sequence selected from Table 1, at least one sense sequence selected from Table 1, or both.

TABLE 1

| | Sense Sequence | Over- | Antisense Sequence | Over- |
|---|---|---|---|---|
| siRNA ID | (5′ - 3′) | hang | (5′ - 3′) | hang |
| CIDEB 31- | CUUACUCAGGUCAGUAUCU | AA | AGAUACUGACCUGAGUAAG | UC |
| 53 | (SEQ ID NO: 2) | | (SEQ ID NO: 50) | |
| CIDEB 450- | AUGUCAAAGCCACAUUCUA | CG | UAGAAUGUGGCUUUGACAU | UC |
| 472 | (SEQ ID NO: 3) | | (SEQ ID NO: 51) | |
| CIDEB 480- | CUAUGAGUUGUGACUUUCA | AG | UGAAAGUCACAACUCAUAG | AG |
| 502 | (SEQ ID NO: 4) | | (SEQ ID NO: 52) | |
| CIDEB 481- | UAUGAGUUGUGACUUUCAA | UU | UUGAAAGUCACAACUCAUA | GA |
| 503 | (SEQ ID NO: 5) | | (SEQ ID NO: 53) | |
| CIDEB 709- | GACUAUGACAGCAUCAAAU | UU | AUUUGAUGCUGUCAUAGUC | UU |
| 731 | (SEQ ID NO: 6) | | (SEQ ID NO: 54) | |
| CIDEB 711- | CUAUGACAGCAUCAAAUUU | CA | AAAUUUGAUGCUGUCAUAG | UC |
| 733 | (SEQ ID NO: 7) | | (SEQ ID NO: 55) | |
| CIDEB 846- | CUGAAGAAUGCUGUUCUUU | CC | AAAGAACAGCAUUCUUCAG | UU |
| 868 | (SEQ ID NO: 8) | | (SEQ ID NO: 56) | |
| CIDEB 894- | CCUGAAAGGCCAAGAUCAA | GA | UUGAUCUUGGCCUUUCAGG | UU |
| 916 | (SEQ ID NO: 9) | | (SEQ ID NO: 57) | |
| CIDEB 1027- | CUUGAGAUCUGUCUUCAUA | CC | UAUGAAGACAGAUCUCAAG | UU |
| 1049 | (SEQ ID NO: 10) | | (SEQ ID NO: 58) | |
| CIDEB 506 | GGCCCAAAGAAAGUACUCA | UU | UGAGUACUUUCUUUGGGCC | UU |
| | (SEQ ID NO: 11) | | (SEQ ID NO: 59) | |
| CIDEB 37 | CAGGUCAGUAUCUAAUAUA | UU | UAUAUUAGAUACUGACCUG | UU |
| | (SEQ ID NO: 12) | | (SEQ ID NO: 60) | |
| CIDEB 943 | GACUGCUGCUACAUCUAAU | UU | AUUAGAUGUAGCAGCAGUC | UU |
| | (SEQ ID NO: 13) | | (SEQ ID NO: 61) | |
| CIDEB 709 | GACUAUGACAGCAUCAAAU | UU | AUUUGAUGCUGUCAUAGUC | UU |
| | (SEQ ID NO: 14) | | (SEQ ID NO: 62) | |
| CIDEB 698 | CCCACUGCAAAGACUAUGA | UU | UCAUAGUCUUUGCAGUGGG | UU |
| | (SEQ ID NO: 15) | | (SEQ ID NO: 63) | |
| CIDEB 472 | GCUCUACUCUAUGAGUUGU | UU | ACAACUCAUAGAGUAGAGC | UU |
| | (SEQ ID NO: 16) | | (SEQ ID NO: 64) | |
| CIDEB 567 | GCCAUAUGUUGCUGGGAAU | UU | AUUCCCAGCAACAUAUGGC | UU |
| | (SEQ ID NO: 17) | | (SEQ ID NO: 65) | |
| CIDEB 29 | GACUUACUCAGGUCAGUAU | UU | AUACUGACCUGAGUAAGUC | UU |
| | (SEQ ID NO: 18) | | (SEQ ID NO: 66) | |
| CIDEB 180 | CCAAAGCAUUGGAGACCCU | UU | AGGGUCUCCAAUGCUUUGG | UU |
| | (SEQ ID NO: 19) | | (SEQ ID NO: 67) | |
| J-004410-06 | AAUAUAAGCUCGGAGUUUG | UU | CAAACUCCGAGCUUAUAUU | UU |
| | (SEQ ID NO: 20) | | (SEQ ID NO: 68) | |
| J-004410-07 | CCGCCUCCAUUCCUACUAA | UU | UUAGUAGGAAUGGAGGCGG | UU |
| | (SEQ ID NO: 21) | | (SEQ ID NO: 69) | |
| J-004410-09 | GAGGAUGACACGUGCCUGA | UU | UCAGGCACGUGUCAUCCUC | UU |
| | (SEQ ID NO: 22) | | (SEQ ID NO: 70) | |
| D-004410-01 | AGGUCAGUAUCUAAUAUAA | UU | UUAUAUUAGAUACUGACCU | UU |
| | (SEQ ID NO: 23) | | (SEQ ID NO: 71) | |
| D-004410-03 | GGAAUUUCCUCCACCCUUC | UU | GAAGGGUGGAGGAAAUUCC | UU |
| | (SEQ ID NO: 24) | | (SEQ ID NO: 72) | |
| D-004410-04 | GAUUCACCUUUGACGUGUA | UU | UACACGUCAAAGGUGAAUC | UU |
| | (SEQ ID NO: 25) | | (SEQ ID NO: 73) | |
| D-004410-05 | UCAGGUCAGUAUCUAAUAU | UU | GAUUCACCUUUGACGUGUA | UU |
| | (SEQ ID NO: 26) | | (SEQ ID NO: 74) | |
| s25891 | CUCUAUGAGUUGUGACUUU | TT | AAAGUCACAACUCAUAGAG | TA |
| | (SEQ ID NO: 27) | | (SEQ ID NO: 75) | |
| s25892 | AGAGGAGGAUGGAACUGCA | TT | UGCAGUUCCAUCCUCCUCU | AG |
| | (SEQ ID NO: 28) | | (SEQ ID NO: 76) | |
| s25893 | GAUUCACCUUUGACGUGUA | TT | UACACGUCAAAGGUGAAUC | GG |
| | (SEQ ID NO: 29) | | (SEQ ID NO: 77) | |
| 121698 | GCCACAUUCUACGGGCUCU | TT | AGAGCCCGUAGAAUGUGGC | TT |
| | (SEQ ID NO: 30) | | (SEQ ID NO: 78) | |
| 122204 | GAAAGUACUCAGGGAGCUC | TT | GAGCUCCCUGAGUACUUUC | TT |
| | (SEQ ID NO: 31) | | (SEQ ID NO: 79) | |
| 122205 | GUCUAUACCCUUACCUGAA | TT | UUCAGGUAAGGGUAUAGAC | TT |
| | (SEQ ID NO: 32) | | (SEQ ID NO: 80) | |
| 46228 | GCUGCUAGCCAAAGCAUUG | TT | CAAUGCUUUGGCUAGCAGC | TC |
| | (SEQ ID NO: 33) | | (SEQ ID NO: 81) | |
| 5581 | GGAGUGGAGUGCUGUCAUA | TT | UAUGACAGCACUCCACUCC | TT |
| | (SEQ ID NO: 34) | | (SEQ ID NO: 82) | |
| 5677 | GGCCAAGAUCAAGAUGUCC | TT | GGACAUCUUGAUCUUGGCC | TT |
| | (SEQ ID NO: 35) | | (SEQ ID NO: 83) | |
| CIDEB 31- | CUUACUCAGGUCAGUAUCU | AA | AGAUACUGACCUGAGUAAG | UC |
| 53 | (SEQ ID NO: 36) | | (SEQ ID NO: 84) | |
| CIDEB 480- | CUAUGAGUUGUGACUUUCA | AG | UGAAAGUCACAACUCAUAG | AG |
| 502 | (SEQ ID NO: 37) | | (SEQ ID NO: 85) | |
| CIDEB 709- | GACUAUGACAGCAUCAAAU | UU | AUUUGAUGCUGUCAUAGUC | UU |
| 731 | (SEQ ID NO: 38) | | (SEQ ID NO: 86) | |
| CIDEB 711- | CUAUGACAGCAUCAAAUUU | CA | AAAUUUGAUGCUGUCAUAG | UC |
| 733 | (SEQ ID NO: 39) | | (SEQ ID NO: 87) | |
| CIDEB 846- | CUGAAGAAUGCUGUUCUUU | CC | AAAGAACAGCAUUCUUCAG | GU |
| 868 | (SEQ ID NO: 40) | | (SEQ ID NO: 88) | |
| CIDEB 894- | CCUGAAAGGCCAAGAUCAA | GA | UUGAUCUUGGCCUUUCAGG | GC |
| 916 | (SEQ ID NO: 41) | | (SEQ ID NO: 89) | |
| CIDEB 1027- | CUUGAGAUCUGUCUUCAUA | CC | UAUGAAGACAGAUCUCAAG | GU |
| 1049 | (SEQ ID NO: 42) | | (SEQ ID NO: 90) | |
| CIDEB 1053- | CCUCAAACUAACAAAAACA | UU | UGUUUUUGUUAGUUUGAGG | GG |
| 1075 | (SEQ ID NO: 43) | | (SEQ ID NO: 91) | |
| CIDEB 1054- | CUCAAACUAACAAAAACAU | UU | AUGUUUUUGUUAGUUUGAG | GG |
| 1076 | (SEQ ID NO: 44) | | (SEQ ID NO: 92) | |
| CIDEB 1056- | CAAACUAACAAAAACAUUU | CC | AAAUGUUUUUGUUAGUUUG | AG |
| 1078 | (SEQ ID NO: 45) | | (SEQ ID NO: 93) | |
| CIDEB 1060- | CUAACAAAAACAUUUCCAA | UA | UUGGAAAUGUUUUUGUUAG | UU |
| 1082 | (SEQ ID NO: 46) | | (SEQ ID NO: 94) | |
| CIDEB 1064- | CAAAAACAUUUCCAAUAAA | AA | UUUAUUGGAAAUGUUUUUG | UU |
| 1086 | (SEQ ID NO: 47) | | (SEQ ID NO: 95) | |
| CIDEB 1075- | CCAAUAAAAAUAUCAAAUA | UU | UAUUUGAUAUUUUUAUUGG | AA |
| 1097 | (SEQ ID NO: 48) | | (SEQ ID NO: 96) | |
| CIDEB 1076- | CAAUAAAAAUAUCAAAUAU | UU | AUAUUUGAUAUUUUUAUUG | GA |
| 1098 | (SEQ ID NO: 49) | | (SEQ ID NO: 97) | |

In some embodiments, double stranded siRNA molecules disclosed herein may have at least one sense sequence having at least about 80% homology (e.g., about 80%, about 85%, about 90%, about 95%, or about 99%) to any one of SEQ ID NOs: 2-49. In some embodiments, double stranded siRNA molecules disclosed herein may have at least one antisense sequence having at least about 80% homology (e.g., about 80%, about 85%, about 90%, about 95%, or about 99%) to any one of SEQ ID NOs: 50-97. In some embodiments, double stranded siRNA molecules disclosed herein may have at least one sense sequence having at least about 80% homology (e.g., about 80%, about 85%, about 90%, about 95%, or about 99%) to any one of SEQ ID NOs: 2-49 and at least one antisense sequence having at least about 80% homology (e.g., about 80%, about 85%, about 90%, about 95%, or about 99%) to any one of SEQ ID NOs: 50-97. In some embodiments, double stranded siRNA molecules disclosed herein may have at least one sense sequence of SEQ ID NOs: 2-49. In some embodiments, double stranded siRNA molecules disclosed herein may have at least one antisense sequence of SEQ ID NOs: 50-97. In some embodiments, double stranded siRNA molecules disclosed herein may have at least one sense sequence of SEQ ID NOs: 2-49 and at least one antisense sequence of SEQ ID NOs: 50-97. In some other aspects, double stranded siRNA molecules disclosed herein may have at least one sense sequence of SEQ ID NOs: 2-19 and at least one antisense sequence of SEQ ID NOS: 50-67. In some aspects, double stranded siRNA molecules disclosed herein may have at least one sense sequence of SEQ ID NOs: 36-49 and at least one antisense sequence of SEQ ID NOs: 84-97. In some aspects, double stranded siRNA molecules disclosed herein may have a sense sequence of SEQ ID NO: 2 and an antisense sequence of SEQ ID NO: 50. In some aspects, double stranded siRNA molecules disclosed herein may have a sense sequence of SEQ ID NO: 3 and an antisense sequence of SEQ ID NO: 51. In some aspects, double stranded siRNA molecules disclosed herein may have a sense sequence of SEQ ID NO: 17 and an antisense sequence of SEQ ID NO: 65. In some aspects, double stranded siRNA molecules disclosed herein may have a sense sequence of SEQ ID NO: 18 and an antisense sequence of SEQ ID NO: 66.

In some embodiments, the present disclosure also encompasses nucleic acid sequences encoding a double stranded siRNA molecule as disclosed herein. In some embodiments, a nucleic acid sequence encoding the double stranded siRNA as disclosed herein may be a double stranded DNA, single stranded DNA, a plasmid vector, a viral vector for example a retroviral vector, lentiviral vector, a pox viral vector, an adenoviral vector, or an adeno-associated viral vector. In some embodiments, the nucleic acid may encode at least one sense sequence having at least about 80% homology (e.g., about 80%, about 85%, about 90%, about 95%, or about 99%) to any one of SEQ ID NOs: 2-49. In some embodiments, the nucleic acids may encode at least one antisense sequence having at least about 80% homology (e.g., about 80%, about 85%, about 90%, about 95%, or about 99%) to any one of SEQ ID NOs: 50-97. In some embodiments, the nucleic acids may encode at least one sense sequence having at least about 80% homology (e.g., about 80%, about 85%, about 90%, about 95%, or about 99%) to any one of SEQ ID NOs: 2-49 and at least one antisense sequence having at least about 80% homology (e.g., about 80%, about 85%, about 90%, about 95%, or about 99%) to any one of SEQ ID NOs: 50-97. In some embodiments, the nucleic acids may encode at least one sense sequence of any one of SEQ ID NOs: 2-49. In some embodiments, the nucleic acids may encode at least one antisense sequence of any one of SEQ ID NOs: 50-97. In some embodiments, the nucleic acids may encode at least one sense sequence of SEQ ID NOs: 2-49 and at least one antisense sequence of SEQ ID NOs: 50-97. In some embodiments, the nucleic acid may encode a sense sequence of SEQ ID NO: 2 and an antisense sequence of SEQ ID NO: 50. In some aspects, the nucleic acid may encode a sense sequence of SEQ ID NO: 3 and an antisense sequence of SEQ ID NO: 51. In some aspects, nucleic acid molecule may encode a sense sequence of SEQ ID NO: 17 and an antisense sequence of SEQ ID NO: 65. In some aspects, nucleic acid molecule may encode a sense sequence of SEQ ID NO: 18 and an antisense sequence of SEQ ID NO: 66.

The present disclosure also provides for methods of making the siRNA molecules disclosed herein. Making of siRNAs may be through chemical synthesis or siRNA may be encoded by a plasmid and transcribed or may be vectored by a virus engineered to express the siRNA. A siRNA may be a single stranded molecule with complementary sequences that self-hybridize into duplexes with hairpin loops. siRNA can also be generated by cleavage of parent dsRNA through the use of an appropriate enzyme such as E. coli RNase III or Dicer. A parent dsRNA may be any double stranded RNA duplex from which a siRNA may be produced, such as a full or partial mRNA transcript. Use of cell lysates or in vitro processing may further involve the subsequent isolation of the short, nucleotide siRNAs (e.g., about 2-25 nucleotides in length) from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse and can be easily adapted to synthesis different siRNAs.

In certain embodiments, siRNA molecules disclosed herein may abolish gene expression of CIDEB. Methods known in the art for the detection and quantification of RNA expression suitable for use herein can include, but are not limited to northern blotting and in situ hybridization, RNAse protection assays, polymerase chain reaction (PCR), reverse transcription polymerase chain reaction (RT-PCR), real-time quantitative reverse transcription PCR (RT-qPCR or qPCR), sequencing-based gene expression analysis (e.g., Serial Analysis of Gene Expression (SAGE)), gene expression analysis by massively parallel signature sequencing (MPSS), and the like.

The siRNA molecules of the present invention may specifically downregulate gene expression of CIDEB or a variant thereof. The term downregulating may be interchangeably expressed as reducing, inhibiting, preventing, blocking or silencing. Here, the phrase “downregulating gene expression” refers to any reduced level of gene expression comparing with an ordinary expression level. For example, the reduced level of gene expression can be from about 70% to 0% of the ordinary expression level. In other words, about 30% to 100% gene expression is downregulated, reduced, blocked, inhibited, prevented or silenced, comparing to the ordinary expression level. Specifically, the reduced level of gene expression is about 70%, 65%, 60%, 55%, 50%, 55%, 50%, 45%, 40%, 35%, 30%, 25%, 20%, 15%, 10%, 8%, 5%, 4%, 3%, 2%, 1% or 0% of the ordinary expression level of CIDEB. The reduced level of gene expression can also be any percentage or range as recited above. On the other hand, the term “specific” or “specifically” used in combination with downregulating refers to downregulation of a target gene's expression with minimal or no binding or downregulation of other nucleic acids or their expressions.

In some embodiments, siRNA molecules disclosed herein may reduce gene expression of CIDEB by at least about 50%. In some aspects, siRNA molecules disclosed herein may reduce gene expression of CIDEB by about 50% to about 99%, about 55% to about 98%, or about 60% to about 95%. In some aspects, siRNA molecules disclosed herein may reduce gene expression of CIDEB by about 50%, about 55%, about 60%, about 65%, about 70%, about 75%, about 80%, about 85%, about 90%, about 95%, or about 99%.

In certain embodiments, siRNA molecules disclosed herein may abolish protein expression of CIDEB. Methods known in the art for the detection and quantification of protein expression suitable for use herein can include, but are not limited to ELISAs (enzyme-linked immunosorbent assays), immunoblot assays, flow cytometric assays, immunohistochemical assays, radioimmuno assays, Western blot assays, an immunofluorescent assays, chemiluminescent assays, mass spectrometry assays, matrix-assisted laser desorption/ionization time-of-flight (MALDI-TOF) mass mapping, liquid chromatography/quadrupole time-of-flight electrospray ionization tandem mass spectrometry (LC/Q-TOF-ESI-MS/MS), two-dimensional polyacrylamide gel electrophoresis (2D-PAGE), and the like.

In some embodiments, siRNA molecules disclosed herein may reduce protein expression of CIDEB by at least about 50%. In some aspects, siRNA molecules disclosed herein may reduce protein of CIDEB by about 50% to about 99%, about 55% to about 98%, or about 60% to about 95%. In some aspects, siRNA molecules disclosed herein may reduce protein expression of CIDEB by about 50%, about 55%, about 60%, about 65%, about 70%, about 75%, about 80%, about 85%, about 90%, about 95%, or about 99%.

In some embodiments, siRNA molecules disclosed herein may have one or more chemical modifications. Non-limiting examples of chemical modifications can include terminal cap moieties, phosphate backbone modifications, and the like. Examples of classes of terminal cap moieties include, without limitation, inverted deoxy abasic residues, glyceryl modifications, 4′,5′-methylene nucleotides, 1-(β-D-erythrofuranosyl) nucleotides, 4′-thio nucleotides, carbocyclic nucleotides, 1,5-anhydrohexitol nucleotides, L-nucleotides, α-nucleotides, modified base nucleotides, threo pentofuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3 aminopropyl phosphate, 6-aminohexyl phosphate, 1,2-aminododecyl phosphate, hydroxypropyl phosphate, 1,4-butanediol phosphate, 3′-phosphoramidate, 5′ phosphoramidate, hexylphosphate, aminohexyl phosphate, 3′-phosphate, 5′-amino, 3′-phosphorothioate, 5′-phosphorothioate, phosphorodithioate, and bridging or non-bridging methylphosphonate or 5′-mercapto moieties. Non-limiting examples of phosphate backbone modifications (i.e., resulting in modified internucleotide linkages) include phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, and alkylsilyl substitutions. Such chemical modifications can occur at the 5′-end and/or 3′-end of the sense strand, antisense strand, or both strands of the siRNA.

Chemical modification of a siRNA molecule disclosed herein may comprise modification of at least one ribosugar moiety of its nucleotide sequence. The ribosugar moiety may be modified with 2 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, 2′-C-allyl, or any combination thereof. In some aspects, a siRNA molecule disclosed herein may have less than about 10% to about 70% (e.g., about 10%, about 20%, about 30%, about 40%, about 50%, about 60%, about 70%) of ribosugar moieties of the total nucleotide sequence modified.

Chemical modification of a siRNA molecule disclosed herein may comprise attaching a conjugate to the siRNA molecule. The type of conjugate used and the extent of conjugation to the siRNA can be evaluated for improved pharmacokinetic profiles, bioavailability, and/or stability of the siRNA while retaining activity. As such, one skilled in the art can screen siRNA molecules having various conjugates attached thereto to identify siRNA conjugates having improved properties using any of a variety of well-known in vitro cell culture or in vivo animal models including the negative-controlled expression studies described above. The conjugate can be attached at the 5′- and/or the 3′-end of the sense and/or the antisense strand of the siRNA via a covalent attachment such as a nucleic acid or non-nucleic acid linker. The conjugate can be attached to the siRNA through a carbamate group or other linking group (see, e.g., U.S. Patent Publication Nos. 20050074771, 20050043219, and 20050158727, the content of each of which is incorporated by reference herein in its entirety). A conjugate may be added to siRNA for any of a number of purposes. For example, the conjugate may be a molecular entity that facilitates the delivery of siRNA into a cell or may be a molecule that comprises a drug or label. Examples of conjugate molecules suitable for attachment to siRNA of the present invention include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof. Other examples include the lipophilic moiety, vitamin, polymer, peptide, protein, nucleic acid, small molecule, oligosaccharide, carbohydrate cluster, intercalator, minor groove binder, cleaving agent, and cross-linking agent conjugate molecules described in e.g., U.S. Patent Publication Nos. 20050119470 and 20050107325, the content of each of which is incorporated by reference herein in its entirety. Other examples include the 2′-O-alkyl amine, 2′-O-alkoxyalkyl amine, polyamine, C5-cationic modified pyrimidine, cationic peptide, guanidinium group, amidininium group, cationic amino acid conjugate molecules, and the like. Additional examples of conjugate molecules include a hydrophobic group, a membrane active compound, a cell penetrating compound, a cell targeting signal, an interaction modifier, or a steric stabilizer as described in U.S. Patent Publication No. 20040167090, incorporated by reference herein in its entirety.

In certain embodiments, siRNA molecules disclosed herein may be conjugated to at least one targeting ligand. Targeting ligands contemplated herein include ligands suitable for targeting siRNA molecules to a liver, a liver tissue, and/or a liver cell. Non-limiting examples of targeting ligands suitable for use herein may include galactose, galactosamine, N-formyl-galactosamine, N-acetylgalactosamine, N-propionyl-galactosamine, N-n-butanoyl-galactosamine, N-iso-butanoylgalactos-amine, galactose cluster, and N-acetylgalactosamine trimer and may optionally have a pharmacokinetic modulator selected from the group consisting of: hydrophobic group having 16 or more carbon atoms, hydrophobic group having 16-20 carbon atoms, palmitoyl, hexadec-8-enoyl, oleyl, (9E, 12E)-octadeca-9, 12dienoyl, dioctanoyl, and C16-C20 acyl, and cholesterol. In some aspects, a liver targeting ligand suitable for use herein may be a N-Acetylgalactosamine (GalNAc) conjugate. In some embodiments, siRNA molecules disclosed herein may be conjugated to at least one GalNAc conjugate. In some embodiments, siRNA molecules disclosed herein may be conjugated to about 1 to about 10 GalNAc conjugates, about 2 to about 9 GalNAc conjugates, or about 3 to about 8 GalNAc conjugates. In some embodiments, siRNA molecules disclosed herein may be conjugated to about 1, about 2, about 3, about 4, about 5, about 6, about 7, about 8, about 9, or about 10 GalNAc conjugates.

Any of the siRNA molecule disclosed herein may target the open reading frame or the 5′ or 3′ UTRs of the CIDEB gene or at least one variant thereof. In some embodiments, at least one variant of CIDEB may comprise rs12590407 G>A, rs368997599 G>A, or any combination thereof. In some aspects, siRNA molecules disclosed herein can specifically downregulate gene expression of CIDEB a variant thereof, such as rs12590407 G>A, rs368997599 G>A, or any combination thereof. The CIDEB gene and/or its variant may be associated with a metabolic disorder such as diabetes, liver disease, liver disfunction, liver injury, and/or a liver damage. The liver disease may comprise fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer, or any combination thereof. Liver damage may include cirrhosis, chronic infection of hepatitis B virus (HBV), chronic infection of hepatitis C virus (HCV), non-alcoholic fatty liver disease (NAFLD), non-alcoholic steatohepatitis (NASH), primary biliary cirrhosis (PBC), hereditary hemochromatosis, type 2 diabetes, obesity, tobacco use, alcohol abuse, long-term anabolic steroid use, tyrosinemia, alpha1-antitrypsin deficiency, porphyria cutanea tarda, glycogen storage diseases, Wilson disease, or any combination thereof.

In certain embodiments, methods and compositions provided herein can include a vector containing any one of the siRNA molecules disclosed herein. In some embodiments, a vector for use herein can be a viral vector. As used herein, the term “viral vector” can refer to a nucleic acid vector construct that includes at least one element of viral origin and has the capacity to be packaged into a viral vector particle and encodes at least an exogenous polynucleotide. In certain embodiments, the vector and/or particle can be utilized for the purpose of transferring any nucleic acids into cells either in vitro or in vivo. Numerous viral vectors are known in the art. The term virion can refer to a single infective viral particle. “Viral vector”, “viral vector particle” and “viral particle” also refer to a complete virus particle with its DNA or RNA core and protein coat as it exists outside the cell. Non-limiting examples of viral vectors for use herein can include adenoviruses, adeno-associated viruses (AAV), herpesviruses, retroviruses, lentiviruses, integrase defective lentiviruses (IDLV), and the like. In some embodiments, a viral vector disclosed herein can be a lentiviral vector. Examples of lentiviruses include, but are not limited to, human lentiviruses such as HIV (in particular HIV-1 or HIV-2), simian immunodeficiency virus (SIV), equine infectious anemia virus (EIAV), feline immunodeficiency virus (FIV), Caprine Arthritis Encephalitis Virus (CAEV), visna and progressive pneumonia viruses of sheep, baboon pseudotype viruses, bovine immunodeficiency virus (BIV), and the like. In some embodiments, siRNA molecules and/or vectors described herein can be prepared by conventional recombinant technology known to one of skill in the art. In other embodiments, siRNA molecules and/or vectors described herein can be prepared by a gene editing method known in the art (e.g., by CRISPR). In certain embodiments, methods provided herein can include generating a cell to express any of the siRNA molecules and/or vectors described herein. In some embodiments, vectors, viral particles, and the like as contemplated herein may be encapsulated into a liposome for delivery to a subject.

In some aspects, the current disclosure also encompasses use of gene editing systems for example CRISPR based systems for abolishing or downregulating gene expression of CIBED. As such, the current disclosure also encompasses compositions comprising a nucleic acid sequence related to a cluster regularly interspaced short palindromic repeats (CRISPR) system, for example a single guide RNA (sgRNA), a CRISPR-RNA (crRNA), a trans-activating crRNA (tracrRNA), and further a plasmid DNA (pDNA) or a viral vector encoding nucleic acid sequence related to a cluster regularly interspaced short palindromic repeats (CRISPR) system, that specifically target CIBED or variants thereof. In some aspects, the gene editing system comprises at least an sgRNA targeting CIBED and an RNA guided endonuclease for example Cas9. In some aspects, the gene editing system comprises at least an sgRNA targeting CIBED and an RNA guided endonuclease for example Cas9.

### (b) Pharmaceutical Compositions

The siRNA molecules targeting CIDEB disclosed herein for use according to the methods herein described may be provided per se and/or as part of a pharmaceutical composition, where modulators and/or inhibitors can be mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism. Herein the term “active ingredient” refers to any of the siRNA molecules disclosed herein. The term “active ingredient” as used herein can also include any vector, medium, microorganism, or cell culture wherein the siRNA molecule is synthesized, expressed and/or contained, such as a genetically modified cell, viral vector, plasmid, bacteria, yeast, fungus, and the culture or medium thereof.

### (i) Pharmaceutically Acceptable Carriers and Excipients

Hereinafter, the phrases “physiologically acceptable carrier” and “pharmaceutically acceptable carrier” are interchangeably used herein to refer to a carrier or a diluent that does not cause significant irritation to an organism and does not abrogate the biological activity and properties of the administered compound. An adjuvant is included under these phrases.

In certain embodiments, compositions disclosed herein may further compromise one or more pharmaceutically acceptable diluent(s), excipient(s), and/or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art.

In some embodiments, pharmaceutical compositions herein may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, or any combination thereof. Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “Remington's Pharmaceutical Sciences,” Mack Publishing Co., Easton, Pa., latest edition, which is incorporated herein by reference.

In certain embodiments, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In some embodiments, any of the well-known techniques, carriers, and excipients may be used as suitable and/or as understood in the art.

In certain embodiments, pharmaceutical compositions described herein may be an aqueous suspension comprising one or more polymers as suspending agents. In some embodiments, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In some embodiments, pharmaceutical compositions disclosed herein may comprise at least about 5%, at least about 10%, at least about 20%, at least about 25%, at least about 30%, at least about 35%, at least about 40%, at least about 45%, or at least about 50% total amount of polymers as suspending agent(s) by total weight of the composition. In some embodiments, pharmaceutical compositions disclosed herein may comprise about 5% to about 99%, about 10%, about 95%, or about 15% to about 90% total amount of polymers as suspending agent(s) by total weight of the composition.

In certain embodiments, pharmaceutical compositions disclosed herein may comprise a viscous formulation. In some embodiments, viscosity of composition herein may be increased by the addition of one or more gelling or thickening agents. In some embodiments, compositions disclosed herein may comprise one or more gelling or thickening agents in an amount to provide a sufficiently viscous formulation to remain on treated tissue. In some embodiments, pharmaceutical compositions disclosed herein may comprise at least about 5%, at least about 10%, at least about 20%, at least about 25%, at least about 30%, at least about 35%, at least about 40%, at least about 45%, or at least about 50% total amount of gelling or thickening agent(s) by total weight of the composition. In some embodiments, pharmaceutical compositions disclosed herein may comprise about 5% to about 99%, about 10%, about 95%, or about 15% to about 90% total amount of gelling or thickening agent(s) by total weight of the composition. In some embodiments, suitable thickening agents for use herein can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or any combination thereof.

In certain embodiments, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some embodiments, pharmaceutical compositions disclosed herein may comprise at least about 5%, at least about 10%, at least about 20%, at least about 25%, at least about 30%, at least about 35%, at least about 40%, at least about 45%, or at least about 50% total amount of one or more agents by total weight of the composition. In some embodiments, pharmaceutical compositions disclosed herein may comprise about 5% to about 99%, about 10%, about 95%, or about 15% to about 90% total amount of one or more agents by total weight of the composition. In some embodiments, one or more of these agents may be added to improve the performance, efficacy, safety, shelf-life and/or other property of the muscarinic antagonist composition of the present disclosure. In some embodiments, additives may be biocompatible, without being harsh, abrasive, and/or allergenic.

In certain embodiments, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some embodiments, any pharmaceutically acceptable organic or inorganic acid may be used. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more acidifying agents by total weight of the composition. In some embodiments, pharmaceutical compositions disclosed herein may comprise about 5% to about 99%, about 10%, about 95%, or about 15% to about 90% total amount of one or more acidifying agents by total weight of the composition.

In certain embodiments, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some embodiments, any pharmaceutically acceptable organic or inorganic base can be used. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more alkalizing agents by total weight of the composition. In some embodiments, pharmaceutical compositions disclosed herein may comprise about 5% to about 99%, about 10%, about 95%, or about 15% to about 90% total amount of one or more alkalizing agents by total weight of the composition.

In certain embodiments, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate, sodium metabisulfite and other materials known to one of ordinary skill in the art. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more antioxidants by total weight of the composition. In some embodiments, pharmaceutical compositions disclosed herein may comprise about 5% to about 99%, about 10%, about 95%, or about 15% to about 90% total amount of one or more antioxidants by total weight of the composition.

In certain embodiments, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some embodiments, any pharmaceutically acceptable organic or inorganic buffer can be used. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more buffering agents by total weight of the composition. In some embodiments, pharmaceutical compositions disclosed herein may comprise about 5% to about 99%, about 10%, about 95%, or about 15% to about 90% total amount of one or more buffering agents by total weight of the composition.

In some embodiments, the amount of one or more buffering agents may depend on the desired pH level of a composition. In some embodiments, pharmaceutical compositions disclosed herein may have a pH of about 6 to about 9. In some embodiments, pharmaceutical compositions disclosed herein may have a pH greater than about 8, greater than about 7.5, greater than about 7, greater than about 6.5, or greater than about 6.

In certain embodiments, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some embodiments, any pharmaceutically acceptable preservative can be used. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more preservatives by total weight of the composition. In some embodiments, pharmaceutical compositions disclosed herein may comprise about 5% to about 99%, about 10%, about 95%, or about 15% to about 90% total amount of one or more preservatives by total weight of the composition.

In certain embodiments, pharmaceutical compositions disclosed herein may comprise one or more surface-acting reagents or detergents. In some embodiments, surface-acting reagents or detergents may be synthetic, natural, or semi-synthetic. In some embodiments, compositions disclosed herein may comprise anionic detergents, cationic detergents, zwitterionic detergents, ampholytic detergents, amphoteric detergents, nonionic detergents having a steroid skeleton, or a combination thereof. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more surface-acting reagents or detergents by total weight of the composition. In some embodiments, pharmaceutical compositions disclosed herein may comprise about 5% to about 99%, about 10%, about 95%, or about 15% to about 90% total amount of one or more surface-acting reagents or detergents by total weight of the composition.

In certain embodiments, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more stabilizers by total weight of the composition. In some embodiments, pharmaceutical compositions disclosed herein may comprise about 5% to about 99%, about 10%, about 95%, or about 15% to about 90% total amount of one or more stabilizers by total weight of the composition.

In some embodiments, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In some embodiments, a vapor pressure depression method is used to calculate the osmolarity of the compositions disclosed herein. In some embodiments, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In some embodiments, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a pharmaceutical composition described herein may have an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In some embodiments, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition. In some embodiments, pharmaceutical compositions disclosed herein may comprise about 5% to about 99%, about 10%, about 95%, or about 15% to about 90% total amount of one or more tonicity modifiers by total weight of the composition.

### (ii) Dosage Formulations

In certain embodiments, the present disclosure provides compositions formulated for one or more routes of administration. Suitable routes of administration may, for example, include oral, rectal, transmucosal, transnasal, intestinal, and/or parenteral delivery. In some embodiments, compositions herein formulated can be formulated for parenteral delivery. In some embodiments, compositions herein formulated can be formulated intramuscular, subcutaneous, intramedullary, intravenous, intraperitoneal, and/or intranasal injections.

In certain embodiments, one may administer a composition herein in a local or systemic manner, for example, via local injection of the pharmaceutical composition directly into a tissue region of a patient. In some embodiments, a pharmaceutical composition disclosed herein can be administered parenterally, e.g., by intravenous injection, intracerebroventricular injection, intra-cisterna magna injection, intra-parenchymal injection, or a combination thereof. In some embodiments, a pharmaceutical composition disclosed herein can administered to subject as disclosed herein. In some embodiments, a pharmaceutical composition disclosed herein can administered to human patient. In some embodiments, a pharmaceutical composition disclosed herein can administered to a human patient via at least two administration routes. In some embodiments, the combination of administration routes by be intracerebroventricular injection and intravenous injection; intrathecal injection and intravenous injection; intra-cisterna magna injection and intravenous injection; and/or intra-parenchymal injection and intravenous injection.

In certain embodiments, pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

In certain embodiments, pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen. For injection, the active ingredients of a pharmaceutical composition herein may be formulated in aqueous solutions, preferably in physiologically compatible buffers such as Hank's solution, Ringer's solution, physiological salt buffer, or any combination thereof.

In certain embodiments, pharmaceutical compositions described herein may be formulated in the form of a nanoparticle. The nanoparticle may have a monolayer enclosing the nanoparticle core, wherein the siRNA molecule is disposed within the nanoparticle core. In an embodiment, the nanoparticle core includes a solid lipid (i.e., lipid that remains solid at room temperature and body temperature) or a liquid lipid (i.e., oil, which remains liquid at room temperature and body temperature, for example, vegetable oil or a lipid extracted from human adipose tissue). In particular, embodiments of the present disclosure include nanoparticles and compositions for the controlled and/or sustained release (e.g., release at a predetermined rate to maintain a certain concentration for a certain period of time) of an agent, such as a small interfering RNA (siRNA) from the nanoparticle.

In certain embodiments, pharmaceutical compositions described herein may be formulated for parenteral administration, e.g., by bolus injection or continuous infusion. Formulations for injection herein may be presented in unit dosage form, e.g., in ampoules or in multidose containers with optionally, an added preservative. In some embodiments, compositions herein may be suspensions, solutions or emulsions in oily or aqueous vehicles, and/or may contain formulatory agents such as suspending, stabilizing and/or dispersing agents.

In certain embodiments, pharmaceutical compositions herein formulated for parenteral administration may include aqueous solutions of the active preparation (e.g., a siRNA molecule) in water-soluble form. In some embodiments, compositions herein comprising suspensions of the active preparation may be prepared as oily or water-based injection suspensions. Suitable lipophilic solvents and/or vehicles for use herein may include, but are not limited to, fatty oils such as sesame oil, or synthetic fatty acids esters such as ethyl oleate, triglycerides or liposomes. In some embodiments, compositions herein comprising aqueous injection suspensions may contain substances which increase the viscosity of the suspension, such as sodium carboxymethyl cellulose, sorbitol, and/or dextran. In some embodiments, compositions herein comprising a suspension may also contain one or more suitable stabilizers and/or agents which increase the solubility of the active ingredients (e.g., a siRNA molecule) to allow for the preparation of highly concentrated solutions.

In some embodiments, compositions herein may comprise the active ingredient in a powder form for constitution with a suitable vehicle, e.g., sterile, pyrogen-free water-based solution, before use.

Pharmaceutical compositions suitable for use in context of the present disclosure may include compositions wherein the active ingredients can be contained in an amount effective to achieve the intended purpose. In some embodiments, a therapeutically effective amount means an amount of active ingredients (e.g., a siRNA molecule) effective to prevent, slow, alleviate or ameliorate symptoms of a disorder (e.g., liver disease) or prolong the survival of the subject being treated.

Determination of a therapeutically effective amount is well within the capability of those skilled in the art, especially in light of the detailed disclosure provided herein.

For any preparation used in the methods of the present disclosure, the therapeutically effective amount or dose can be estimated initially from in vitro and cell culture assays and or screening platforms disclosed herein. For example, a dose can be formulated in animal models to achieve a desired concentration or titer. Such information can be used to more accurately determine useful doses in humans.

In some embodiments, toxicity and therapeutic efficacy of the active ingredients disclosed herein can be determined by standard pharmaceutical procedures in vitro, in cell cultures or experimental animals. In some embodiments, data obtained from these in vitro and cell culture assays and animal studies can be used in formulating a range of dosage for use in a human subject. In some embodiments, a dosage for use herein may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1).

In certain embodiments, dosage amounts and/or dosing intervals may be adjusted individually to brain or blood levels of the active ingredient that are sufficient to induce or suppress the biological effect (minimal effective concentration, MEC). In some embodiments, the MEC for an active ingredient (e.g., a siRNA molecule or composition disclosed herein) may vary for each preparation but can be estimated from in vitro data. In some embodiments, dosages necessary to achieve the MEC herein may depend on individual characteristics and route of administration. Detection assays can be used to determine plasma concentrations.

In certain embodiments, depending on the severity and responsiveness of the condition to be treated, dosing with compositions herein can be of a single or a plurality of administrations, with course of treatment lasting from several days to several weeks or until cure is affected or diminution of the disease state is achieved.

In certain embodiments, amounts of a composition herein to be administered will be dependent on the subject being treated, the severity of the affliction, the manner of administration, the judgment of the prescribing physician, and the like. In some embodiments, effective doses may be extrapolated from dose-responsive curves derived from in vitro or in vivo test systems.

### III. Methods of Use

The present disclosure provides for methods of treating, attenuating, and preventing liver disease in a subject in need thereof. In several embodiments, a method for treating, attenuating, or preventing liver disease in a subject can include administering to a subject, including a human subject, an effective amount of one or more siRNA molecules targeting CIDEB disclosed herein, or a nucleic acid encoding an siRNA molecule targeting CIBED as disclosed herein. In several embodiments, a method for treating, attenuating, or preventing liver disease in a subject can include administering to a subject, including a human subject, an effective amount of a nucleic acid encoding a suitable sgRNA, or a suitable sgRNA targeting CIBED and a RNA guided endonuclease

Methods disclosed herein may include treating a subject in need thereof by administrating a therapeutically effective amount of one or more siRNA molecules or a pharmaceutical composition disclosed herein. The subject may be a human subject having or suspected of having, or at risk of having liver disease, liver damage, liver dysfunction, liver injury. The term “liver disease”, “liver injury” or “liver dysfunction” may be used interchangeably and refer to any injury of the liver, including but not limited to hardening of the liver, scarring of the liver, decreased or abnormal biliary tract function, abnormal liver enzyme activity, cirrhosis of the liver, abnormal physiology as determined by common diagnostic methods include but not limited to ultrasound, or biopsy/histopathology, necrosis of the liver and the like. Non-limiting examples of liver disease to be treated using the methods disclosed herein may include fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer, HCC, or any combination thereof. In some embodiments, the subject benefits of an increase in life expectancy compared to an untreated subject with identical disease condition and predicted outcome. In some other embodiments, the treatment improves the subject's liver function as compared to an untreated subject with identical disease condition and predicted outcome. In yet other embodiments, the treatment attenuates the subject's liver fibrosis as compared to an untreated subject with identical disease condition and predicted outcome. In some embodiments, the treatment prevents additional liver fibrosis in the subject compared to an untreated subject with identical disease condition and predicted outcome.

A subject suitable for the liver disease treatment as disclosed herein used herein may be selected based on the subject's diagnosis. In some embodiments, a method of diagnosis may detect one or more serum markers indicative of liver disease. Non-limiting examples of serum markers indicative of a liver disease (e.g., NAFLD, NASH, or HCC) may include alpha-fetoprotein (AFP) (e.g., an AFP level of 20 ng/ml or higher), des-gamma-carboxy prothrombin, lens culinaris agglutinin-reactive AFP (AFP-L3), and the like. The diagnosis method may also include the evaluation of at least one clinical symptom associated with a liver disease. Non-limiting examples of clinical symptoms associated with a liver disease may include mild to moderate upper abdominal pain, weight loss, early satiety, or a palpable mass in the upper abdomen, paraneoplastic syndrome, hypoglycemia, erythrocytosis, hypercalcemia, intractable diarrhea and associated electrolyte disturbances (e.g., hyponatremia, hypokalemia, metabolic alkalosis), cutaneous manifestations (e.g., dermatomyositis, pemphigus foliaceus, seborrheic keratosis, pityriasis rotunda), intraperitoneal bleeding, jaundice, fever, pyogenic liver abscess, and the like. Other aspects of diagnosis may include at diagnosis and/or a determination of severity of cirrhosis, chronic infection of hepatitis B virus (HBV), chronic infection of hepatitis C virus (HCV), non-alcoholic fatty liver disease (NAFLD), non-alcoholic steatohepatitis (NASH), primary biliary cirrhosis (PBC), hereditary hemochromatosis, type 2 diabetes, obesity, tobacco use, alcohol abuse, long-term anabolic steroid use, tyrosinemia, alpha1-antitrypsin deficiency, porphyria cutanea tarda, glycogen storage diseases, Wilson disease, or any combination thereof.

In some embodiments, a subject can be diagnosed and/or predicted to have high or low risk for a liver disease (e.g., NASH or NAFLD) by histological or imaging-based examinations, such as contrast-enhanced multiphase CT, ultrasound, and/or MRI. Imaging features used to diagnose may include liver size, kinetics, and pattern of contrast enhancement, and growth on serial imaging wherein size may be measured as the maximum cross-section diameter on the image where the lesion is most clearly seen. The histologic appearance of NASH or NAFLD biopsies can include steatosis, inflammation, and fibrosis.

The methods and compositions of the present disclosure are useful for the treatment of subjects having fatty liver related disorders, such as NAFLD and/or NASH. The subject may have normal or substantially normal biliary tract function. Normal or substantially normal biliary tract function may be determined in a subject using any suitable methods known in the art. Generally, preferred tests for biliary tract function in NASH patients may be characterized in two groups: physiological based tests and biochemical based tests. Physiological based tests may include but are not limited to abdominal ultrasound, abdominal CT scan, abdominal MRI scan, endoscopic retrograde cholangiopancreatography (ECRP), Percutaneous transhepatic cholangiogram (PTCA) or Magnetic resonance cholangiopancreatography (MRCP). Biochemical based tests may include but are not limited to GGT tests, liver function tests, bilirubin tests, alkaline phosphatase (ALP) tests, liver enzyme tests, amylase blood test, lipase blood test, prothrombin time, and measurement of urine bilirubin. In most cases, one or more tests may be used to characterize liver function in NASH subjects.

In some embodiments, any of the methods disclosed herein can further include monitoring occurrence of one or more adverse effects in the subject. Adverse effects may include, but are not limited to, hepatic impairment, hematologic toxicity, neurologic toxicity, cutaneous toxicity, gastrointestinal toxicity, or a combination thereof. When one or more adverse effects are observed, the methods disclosed herein can further include reducing or increasing the dose of one or more of the treatment regimens depending on the adverse effect or effects in the subject. For example, when a moderate to severe hepatic impairment is observed in a subject after treatment, compositions of use to treat the subject can be reduced in concentration or frequency.

In certain embodiments, treatments administered according to the methods disclosed herein can improve patient life expectancy compared to the life expectancy of an untreated subject with identical disease condition (e.g., NAFLD or NASH) and predicted outcome. As used herein, “patient life expectancy” is defined as the time at which 50 percent of subjects are alive and 50 percent have passed away. In some embodiments, patient life expectancy can be indefinite following treatment according to the methods disclosed herein. In other aspects, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In some embodiments, patient life expectancy can be increased at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, patient life expectancy can be increased at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated patient with identical disease condition and predicted outcome.

In some embodiments, treatment of a liver disease, such as NAFLD or NASH, according to the methods disclosed herein can result in attenuating, shrinking, reducing or preventing of a liver fibrosis in comparison to the starting size of the liver fibrosis. In some embodiments, liver fibrosis attenuating or shrinking may be at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% (meaning that the liver tumor is completely gone after treatment) compared to the starting degree of liver fibrosis.

In some embodiments, treatment of a liver disease, such as NAFLD or NASH, according to the methods disclosed herein can result in an improved liver function. Liver function may be tested through routine biochemical methods. Biochemical tests may include but are not limited to GGT tests, liver function tests, bilirubin tests, alkaline phosphatase (ALP) tests, liver enzyme tests, amylase blood test, lipase blood test, prothrombin time, and measurement of urine bilirubin. In some cases, one or more tests may be used to characterize biliary function. In some cases, a combination of tests may be used to assess biliary functions. The liver function improvement can also be assessed by subject's symptom relief, such as relieving in fatigue, weight loss, and weakness of the subject. Other relieved symptoms may include reduced fluid retention, muscle wasting, bleeding from the intestines, and any combination thereof.

### IV. Kits

The present disclosure provides kits for using the methods disclosed herein. In some aspects, the present disclosure provides a kit for treating a liver disease (e.g., NASH or NAFLD) as disclosed herein and for diagnosing the liver disease. Such a kit may comprise a means for holding and/or administering such a siRNA composition or a pharmaceutical composition.

In some embodiments, kits disclosed herein can have a medical container, which holds the composition in a safe, stable and durable way. In some examples, kits disclosed herein may also comprise a means to administer the composition, such as a needle or a spatula.

Any of the kits may further comprise an instruction manual providing guidance for using the kit for treatment. The manual may be written with the physician or the liver specialist as the intended reader.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1

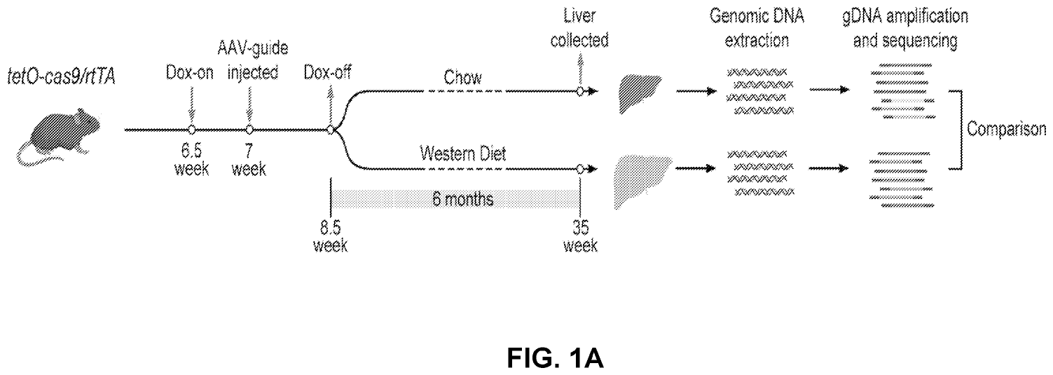

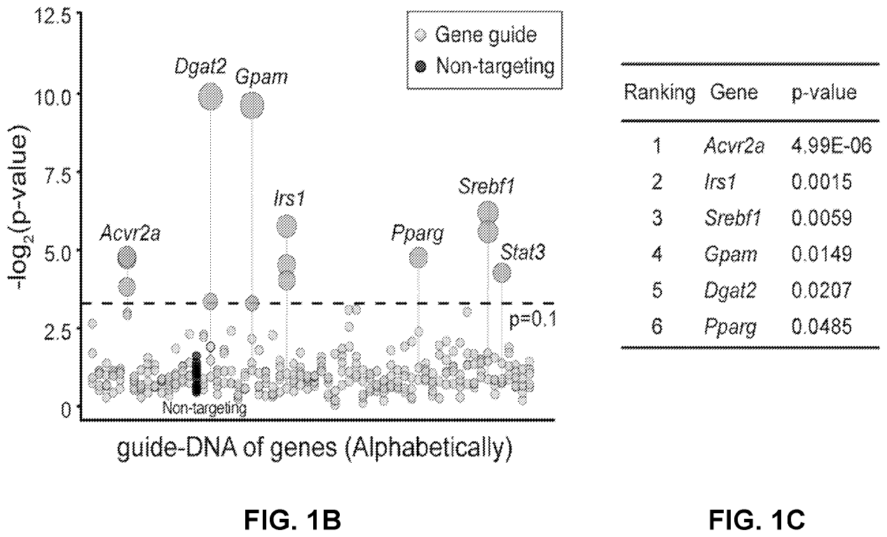

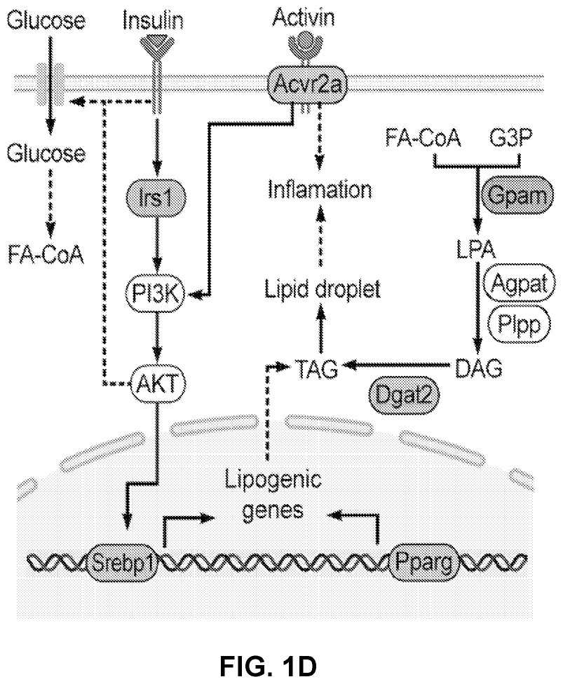

To broadly understand how mutations in NASH candidate genes influence clonal competition in an unbiased fashion in animal models, a method was developed to generate heterogeneous collections of somatically mutant cells within the tissues of Cas9-expressing mice. A hybrid Adeno-Associated Virus (AAV) was created that carries sleeping beauty transposase (SB100), Cre, single guide (sg) RNAs, and whose payload was flanked by transposon integration sequences (SB-IR). This AAV was used to deliver sgRNAs into the livers of dox-inducible Cas9-expressing mice for in vivo CRISPR screening. In brief, a pooled screening of 63 NASH genes identified in GWAS (i.e., APOB, TM6SF2, GCKR), exome-seq (i.e., PNPLA3, MBOAT7, HSD17B13), somatic mutation sequencing efforts (i.e., PPARGC1B, FOXO1, GPAM, ACVR2A) or biochemical studies was performed. Induction of Cas9 expression after AAV injection established a pool of hepatocytes with mosaic deletion of these NASH related genes. These mosaic mice were then given normal chow (NC) or western diet (WD: high sugar, fat, cholesterol) to understand clonal dynamics in both dietary conditions. After 6 months, deep sequencing of sgRNAs pinpointed the genes that had the largest effects on clonal fitness, specifically in the NASH setting. To exclude purely proliferative effects that would occur independent of NASH, enriched or depleted sgRNAs that were shared in NC and WD conditions were excluded. Six genes, when deleted, were most associated with clonal expansion: Acvr2a, Irs1, Srebp1, Gpam, Dgat2, and Pparg (FIGS. 1A-1D). These genes, when inhibited, protect the liver from getting NASH. Two of these genes, Gpam and Acvr2a, were also two of the 5 most frequently mutated genes in human NASH cirrhosis samples. The in vivo genetic screen of the exemplary method herein proved the concept that mutations observed in human cirrhosis, which are selected for during the long course of chronic liver disease, can represent effective drug targets. This functional screen in mice proves instrumental in our understanding of the significance of human somatic mutations in NASH cirrhosis samples.

Another NAFLD gene identified was cell death-inducing DFF45-like effector B (CIDEB) which is mutated in human NASH cirrhosis samples. By way of introduction and not to be bound by theory, the SREBP transcription factors regulate lipid metabolism by controlling the expression of lipogenesis, sterol production, and lipid uptake genes. Complexes of SREBP, SCAP, and the sterol binding protein Insig-1/2 are localized to the ER in the presence of sterols. Upon sterol deprivation, SREBP/SCAP move to the Golgi, where SREBP cleavage occurs to release the N-terminal part of the protein that acts as the transcriptional activator. CIDEB is a regulator of the ER to Golgi transport process and augments the activity of SREBP in this way. CIDEB, an ER and lipid droplet protein expressed in the liver, regulates lipid droplet fusion and VLDL lipidation. Overall, CIDEB coordinates multiple independent avenues of lipid homeostasis. Thus, the observation of loss of function mutations in metabolic factors that generate hepatic lipids, like CIDEB, suggest that some somatic mutations confer increased tissue fitness through a reversal of the driving etiology of disease.

To further ascertain which genes are increasing clonal expansion through an influence on metabolic fitness, conditional knockout mouse models are needed. The CRISPR plus AAV-sgRNA approach described herein was first used to generate liver-wide conditional deletion models. AAV-sgRNAs against GFP or LacZ are also implemented as independent control models. High titer AAVs-sgRNAs against CIDEB are injected into Cas9 expressing mice at 8 weeks of age, then after 2 weeks, mice are given western diet (WD) for three months. For each conditional CRISPR KO model, body weight, liver weight, histology, steatosis, fibrosis, serum tests (liver function tests, cholesterol, TGs, non-esterified free fatty acids (NEFAs)) are measured.

In order to study loss of function CIDEB mutations with while liver deletion or clonal mosaicism using traditional mouse genetic tools, both whole body CIDEB knockout mice as well as CIDEB floxed mice targeting exon 3, which is predicted to lead to frameshift and stop codon causing early termination of the protein, were also generated. Using these mice, it was confirmed that CIDEB floxed mice achieved CIDEB deletion after Cre recombination. Additionally, liver specific CIDEB knockout mice were protected from liver triglyceride accumulation associated with 3 months of chronic WD feeding.

Because it is not currently known if liver specific deletion of CIDEB can mitigate NASH, studies herein examine whether whole liver genetic CIDEB deletion prevents NASH development. In brief, novel CIDEB floxed mice were generated to more rigorously examine liver specific loss of CIDEB in the liver. Use of these mice allow for nearly 100% hepatocyte deletion, which is not achieved using the above CRISPR approaches. Spatial and temporal conditional deletion are achieved with AAV-TBG-Cre, which expresses Cre recombinase only in hepatocytes. Mice are given AAV at 6 weeks and initiated on NASH diets at 8 weeks of age. CIDEB^{fl/fl} and CIDEB^{+/+} mice are given AAV-TBG-Cre to mediate flox recombination. The following 4 groups are compared: CIDEB^{+/+}+AAV-TBG-Cre on normal chow (NC), CIDEB^{fl/fl}+AAV-Cre on NC, CIDEB^{+/+}+AAV-TBG-Cre on WD, CIDEB^{fl/fl}+AAV-TBG-Cre on WD. The NC groups allow for ruling out any phenotypic effects of CIDEB loss that arise independently of diet. These rigorous reference standard KO mice are used to determine the extent to which NASH is prevented. The effects of CIDEB deletion on liver steatosis, inflammation, and fibrosis are characterized after 12 and 24 weeks of NASH diets. In WT control mice fed WD, hepatic steatosis and inflammation are prominent at ˜12 weeks, while hepatocyte ballooning and fibrosis (features that are more NASH specific), are observed by ˜24 weeks. Liver toxicity is monitored prior to liver harvesting (every 12 weeks) by blood AST, ALT, total bilirubin, albumin, and complete blood counts. Plasma and liver triglycerides (TG), non-esterified free fatty acids (NEFA), and cholesterol, which are the critical lipid markers of NASH and metabolic syndrome, are also examined. Harvested livers are sectioned for macro/microscopic evidence of tissue injury, inflammation, and fibrosis. Livers are assessed in a blinded fashion for components of the NASH Activity Score (NAS): steatosis, lobular inflammation, and hepatocyte ballooning. Separately, the degree of fibrosis is quantified.

Currently, there are no attempts to therapeutically target CIDEB in liver disease. As such, exemplary methods herein determine if a siRNA molecule against CIDEB prevents and/or reverses NASH in mice. In brief, an siRNA tool compound was identified to study mouse CIDEB phenotypes. The 8 available Dharmacon siRNAs against mouse Cideb were have tested and the siRNA sequences with the highest knockdown efficiency were identified. These siRNAs are modified and conjugated with GalNAc in the standard fashion. Briefly, nucleolytic degradation and immune responses in vivo are minimized by modifying every 2′-position with 2′-O-methyl. GalNAc conjugation allows for high efficiency delivery to hepatocytes without lipid nanoparticle packaging. The best in vivo dosing regimen for GalNAc-siRNA against CIDEB is then determined. After defining optimal dosing, the ability of GalNAc-siCideb to prevent or reverse NASH is determined. In brief, GalNAc-siRNA SC injections are started at 6 weeks of age and NASH diets are started at 8 weeks and continued for 12 weeks (6 total siRNA doses). Mice are euthanized at 20 weeks of age, a time point at which is used to determine steatosis, inflammation, and fibrosis. To determine if NASH can be reversed, siRNA dosing is initiated in mice that have already received 24 weeks of WD diets (30 weeks of age), and the WD and siRNAs in continued for 12 weeks total. Livers are then assessed for pathological features of NASH. Hepatocyte ballooning, inflammation, and fibrosis, and other features that characterize NASH are also assessed.

### Example 2

Specific siRNA sequences were designed to target CIDEB for the treatment of NASH. Based on the human somatic sequencing data which has identified recurrent loss of function CIDEB mutations in NASH livers and based on what the biology of CIDEB in mouse models, CIDEB represents a promising therapeutic target for human NASH.

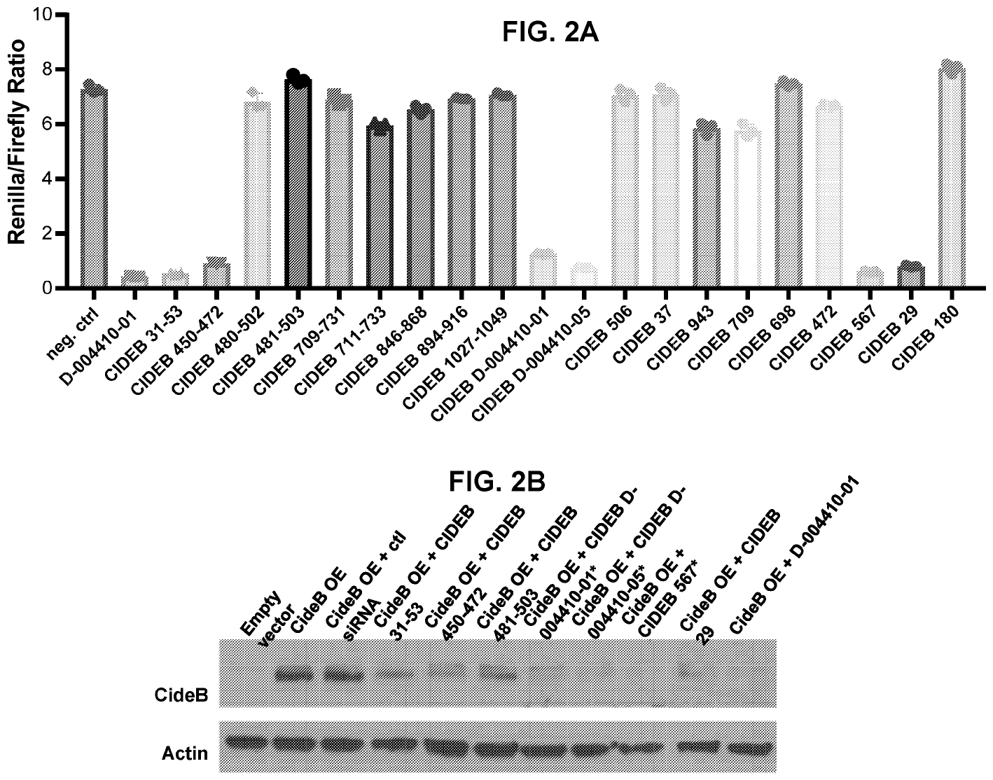

Tests identified optimized siRNA sequences corresponding to the human sequences of human CIDEB. This involved screening many candidate siRNAs per gene target using in vitro luciferase reporter-based assays. In brief, full length CIDEB cDNA (without a 3′UTR) was cloned into a psicheck2 plasmid backbone containing a luciferase gene. This created a Renilla luciferase-CIDEB fusion gene. The luciferase assay is a dual reporter system with Firefly luciferase as a control and Renilla luciferase as the read out for transcription and translation. Thus, the ratio of Renilla to Firefly signal accounts for variations in transfection efficiency and cell viability. The assay was carried out in a 96 well plate format with technical replicates using a 48-hour time point for the assay readout. The reporter along with siRNAs were co-transfected into Cos7 monkey kidney cells using lipofectamine. After 48 hours, the cells were lysed, and the signal was captured by a luminometer using the substrates for Firefly and Renilla. Effective siRNAs against a target gene showed a reduced ratio for Renilla vs. Firefly signal compared to non-targeting controls. A total of 37 siRNAs targeting the entire CIDEB cDNA+3′UTR were designed and tested. siRNAs designed by Dharmacon and ThermoFisher were also tested. At least 17 effective siRNAs were identified against human CIDEB (Table 2). In other words, 17 siRNAs shown in Table 2 had less than 40% of CIDEB mRNA remaining. The siRNAs designed and tested in the exemplary methods herein are shown in FIGS. 2A-2B. Optimized siRNAs are modified by conjugating to acetylgalactosamine (GalNAC) in the standard fashion and tested in vivo.

TABLE 2

| | % mRNA remaining | Experimental |
|---|---|---|
| | (compared to neg. | Design or |
| | ctl siRNA, at 3.3 | Manufacturer |
| siRNA ID | nM or 0.5 pmol) | Design |
| CIDEB 31-53 | 7.6 | Experimental |
| CIDEB 450-472 | 12.6 | Experimental |
| CIDEB 480-502 | 93.6 | Experimental |
| CIDEB 481-503 | 104.8 | Experimental |
| CIDEB 709-731 | 94.6 | Experimental |
| CIDEB 711-733 | 81.8 | Experimental |
| CIDEB 846-868 | 89.7 | Experimental |
| CIDEB 894-916 | 95.3 | Experimental |
| CIDEB 1027-1049 | 97.1 | Experimental |
| CIDEB 506 | 97.0 | Experimental |
| CIDEB 37 | 97.5 | Experimental |
| CIDEB 943 | 80.4 | Experimental |
| CIDEB 709 | 79.2 | Experimental |
| CIDEB 698 | 102.7 | Experimental |
| CIDEB 472 | 91.6 | Experimental |
| CIDEB 567 | 8.4 | Experimental |
| CIDEB 29 | 11.0 | Experimental |
| CIDEB 180 | 110.5 | Experimental |
| J-004410-06 | 28.9 | Dharmacon |
| J-004410-07 | 42.9 | Dharmacon |
| J-004410-09 | 36.5 | Dharmacon |
| D-004410-01 | 6.4 | Dharmacon |
| D-004410-03 | 24.6 | Dharmacon |
| D-004410-04 | 16.0 | Dharmacon |
| D-004410-05 | 7.8 | Dharmacon |
| s25891 | 19.8 | ThermoFisher |
| s25892 | 17.6 | ThermoFisher |
| s25893 | 20.8 | ThermoFisher |
| 121698 | 35.7 | ThermoFisher |
| 122204 | 71.5 | ThermoFisher |
| 122205 | 14.5 | ThermoFisher |
| 46228 | 28.4 | ThermoFisher |
| 5581 | 37.4 | ThermoFisher |
| 5677 | 75.0 | ThermoFisher |

### Example 3

Some of these human siRNAs against CIDEB disclosed herein are tested in human cell line models of NASH. The tests are performed using optimized siRNAs and/or their conjugates with acetylgalactosamine (GalNAc) to improve liver targeting. The experimental testing of candidate siRNAs has three steps. First, the identified siRNAs are used in human liver cancer cells, such as Huh7 or HepG2, to knockdown target genes of interest, e.g., CIDEB. Specifically, Huh7 cells are grown to 60-80% confluence and then transfected with siRNAs. Opti-MEM (Thermo Fisher 31985062) containing the siRNAs and Lipofectamine are combined, incubated, and then added to cells for transfection (400 μl/well to a 6-well plate or 2.4 ml to a 100 mm plate). Transfection medium is replaced after 6 h with medium containing 100 M FA. Then, cells are fed lipids and label with lipid dyes. Lastly, fluorescence-activated cell sorting (FACS) is performed to quantitate lipid accumulation. FACS to purify specific cell populations based on phenotypes detected by flow cytometry. This method enables characterization of a single cell population without the influence of other cells. Cells are stained with 1 ml PBS containing 10 μg of BODIPY 493/503 (BD) (Thermo Fisher D3922) for 15 minutes at room temperature (23° C.±5° C.), then subject to FACs measurements. Early studies show that in lipophilic fluorophore-stained cells, fluorescence intensity measured by FC reflects lipid levels.

An aliquot of the cells grown under each condition is assayed for triacylglycerol (TG). In brief, cells are trypsinized, washed once with PBS, resuspended in PBS with 10 mM EDTA, and then counted. Cells are divided in triplicate into 13×100 mm glass tubes in a final volume of 200 μl. Triolein standards (Sigma T7140) are also prepared in a final volume of 200 μl PBS/10 mM EDTA in 13×100 mm glass tubes. TGs are extracted and quantified. Briefly, 2 ml of isopropanol:hexane:water (40:10:1) is added to cells or standards and the samples are vortexed, covered, and incubated at room temperature for 30 minutes. Then, 500 μl of a 1:1 mixture of hexane:diethylether is added to the samples followed by vortexing and incubating for an additional 10 min at room temperature. Next, 1 ml of water is added to samples, tubes are vortexed, and layers are allowed to separate at room temperature while covered for 30-45 minutes. Using Pasteur pipettes, the top layer is transferred to 12×75 mm glass tubes and dried under N₂ to completion. Following the drying step, 400 μl of Infinity triglyceride reagent (Thermo Scientific TR22421) is added to each tube and vortexed. Tubes are covered and incubated for 90 minutes at 37° C. with shaking at 250 rpm. Finally, 300 μl of each sample is transferred to 96-well plates and absorbance is measured at 540 nm using a microplate reader. To determine whether fixing cells affected the TG measurement, TG is quantified in fixed and unfixed cells from the same batch of Huh7 cells incubated in 2 mM FA.

What is claimed is:

1. A composition comprising a nucleic acid that downregulates expression of Cell Death-Inducing DFF45-like Effector Protein B (CIDEB) or a variant thereof.

2. The composition of claim 1, wherein the nucleic acid that downregulates expression of CIBED comprises a siRNA, a cluster regularly interspaced short palindromic repeats (CRISPR) related nucleic acid, a single guide RNA (sgRNA), a CRISPR-RNA (crRNA), or a trans-activating crRNA (tracrRNA).

3. The composition of claim 2, wherein the nucleic acid that downregulates gene expression of CIDEB or a variant thereof is a small interfering RNA (siRNA) molecule.

4. A composition comprising a plasmid or a viral vector, wherein the plasmid or the viral vector comprises a nucleic acid encoding the siRNA molecule of claim 3.

5. The composition of any one of claim 3 or 4, wherein the siRNA molecule comprises a nucleotide sequence that is 2 to 30 nucleotides in length and is at least 80% homologous to at least 2 to 30 contiguous nucleotides of a human CIDEB cDNA sequence.

6. The composition of claim 5, wherein the human CIDEB cDNA sequence is SEQ ID NO: 1.

7. The composition of any one of claims 1-3, wherein the siRNA molecule targets the open reading frame or the 5′ or 3′ UTRs of the CIDEB gene.

8. The composition of any one of claims 3-7, wherein the siRNA molecule comprises at least one sense sequence, at least one antisense sequence, or at least one sense sequence and at least one antisense sequence.

9. The composition of any one of claims 3-8, wherein the siRNA molecule comprises a nucleotide sequence SEQ ID NOs: 2-97 or any combination thereof.

10. The composition of claim 8, wherein the at least one sense sequence comprises SEQ ID NOs: 2-49.

11. The composition of claim 8, wherein the at least one antisense sequence comprises SEQ ID NOs: 50-95.

12. The composition of claim 2, wherein the nucleic acid that downregulates expression of CIBED is a sgRNA molecule.

13. A composition comprising a plasmid or a viral vector, wherein the plasmid or viral vector comprises a first nucleic acid encoding the sgRNA molecule of claim 12 and optionally a second nucleic acid encoding an RNA guided nuclease.

14. The composition of claim 13, wherein the RNA guided nuclease is a Cas endonuclease.

15. The composition of any one of claims 3-11, wherein the siRNA molecule specifically downregulates gene expression of at least one variant of CIBED.

16. The composition of any one of claims 12-13, wherein the sgRNA molecule specifically downregulates gene expression of at least one variant of CIBED.

17. The composition of any one of claim 15 or 16, wherein the at least one variant of CIDEB is associated with a liver disease.

18. The composition of claim 17, wherein the liver disease comprises fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer, or any combination thereof.

19. The composition of any one of claims 15-18, wherein the at least one variant of CIDEB comprises rs12590407 G>A, rs368997599 G>, or any combination thereof.

20. The composition of any one of claim 1-3 or 12, wherein the nucleic acid molecule is conjugated to least one targeting ligand.

21. The composition of claim 20, wherein the at least one targeting ligand comprises a liver targeting ligand.

22. The composition of claim 21, wherein the liver targeting ligand comprises at least one N-Acetylgalactosamine (GalNAc) conjugate.

23. The composition of claim 22, wherein the siRNA molecule is conjugated to about one to about three GalNAc conjugates.

24. The composition of any one of claim 1-3 or 12, wherein the nucleic acid molecule comprises at least one chemical modification.

25. The composition of of claim 24, wherein the nucleic acid molecule comprises a modification at least one ribosugar moiety of its nucleotide sequence.

26. The composition of claim 25, wherein at least one ribosugar moiety is modified with 2 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, 2′-C-allyl, or any combination thereof.

27. The composition of either claim 25 or claim 26, wherein less than about 10% to about 70% of ribosugar moieties of the total nucleotide sequence is modified.

28. A pharmaceutical composition comprising any one of the compositions of claims 1-27 and at least one pharmaceutically acceptable carrier.

29. The pharmaceutical composition of claim 28, further comprising a nanoparticle.

30. The pharmaceutical composition of either claim 28 or claim 29, further comprising a lipid.

31. A method of for treating a subject in need thereof, the method comprising administrating a therapeutically effective amount of the composition of any one of claims 1-77 or the pharmaceutical composition of any one of claims 28-30.

32. The method of claim 31, wherein the subject in need thereof, is a human subject having or suspected of having a liver disease.

33. The method of claim 32, wherein the liver disease comprises fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer, or any combination thereof.

34. The method of any one of claims 31-33, wherein the method of administering comprises parenteral administration.

35. The method of any one of claims 31-34, wherein administration of a therapeutically effective amount of the composition of any one of claims 1-27 or the pharmaceutical composition of any one of claims 28-30 increases life expectancy of the subject compared to an untreated subject with identical disease condition and predicted outcome.

36. The method of any one of claims 31-35, wherein administration of a therapeutically effective amount of the composition of any one of claims 1-27 or the pharmaceutical composition of any one of claims 28-30 increases liver function of the subject compared to an untreated subject with identical disease condition and predicted outcome.

37. The method of any one of claims 31-36, wherein administration of a therapeutically effective amount of the composition of any one of claims 1-27 or the pharmaceutical composition of any one of claims 28-30 attenuates liver fibrosis in the subject compared to an untreated subject with identical disease condition and predicted outcome.

38. The method of any one of claims 31-37, wherein administration of a therapeutically effective amount of the composition of any one of claims 1-27 or the pharmaceutical composition of any one of claims 28-30 prevents additional liver fibrosis in the subject compared to an untreated subject with identical disease condition and predicted outcome.

39. A kit comprising
(i) a container holding the composition of any one of claims 1-27 or the pharmaceutical composition of any one of claims 28-30;
(ii) a pharmaceutical administrative means; and
(iii) an instruction.


SEQUENCE LISTING (97 sequences)

SEQ ID NO: 1 - DNA, 2215 bp, Homo sapiens
  source 1..2215: mol_type = genomic DNA
cccttccggtggagccagcgctgcgaccgcctgcagaaggttgactgcgtggtagggggcccagagcaagccgaaggcaagcacgatggcgctcaccagccggcccacccgcgccccgtgccgcccggagccccagcgggcgccccgcagccgtgccagcgtcacgctgtagcagccgagcatcagcccgaaaggaagcacgaaagcggtcagagtctccaggctcaggtgggcggcggcgtggaccggcgacgggtggcacagctggcatacgcggtccctccacaggtggcggtagacggcggccgggacggcgagcaacagggcggccagccagaccgccagcagcaggcggcgggccagggccgggctgcgcagccgaggcgccaggaaggggcgggtgactgcgaggcagcgctgcaggctgagcaggccggtgagcagcacgctggcgtacatgctgagcgcgcacacgtagtacaccgccttgcagcccgcctggcccagcggccaggcctgccgggtcaggaaggccacaaagagcggcgtgagcagcagcaccgcgccgtcggccagcgccaggtgcagcacaagcgtggccgccagcggtcgcccccgtgcaggccgccagcccgccaagctccacaccacgaagccgttgccaggcagccccagcagcgccgccagcagcaggaaggctgtgcctgtggcccgcgaagtcttccagctcagcagtgtctcgttccctgggggacggtagcagaccgacatccttctgggcctacagtgagagtctcagcttccatgcaactgtccatcacggctgcaactgaaatcagagctgggacacagcgcaccagaagctaaagtcttgatgccatcaaaggacatccctgccccattcacatctctgtcacgtccactaatcggcaaaaggagaaaagtgagagaagatgacctaagtgtgactgcagcaggcagctctggaaaatgaagccagagcagtgagccagcccctcctccgaccaaggaggaaggaaagagcagccccagcacaggagagaaccacccagcccagaagttccagggaaggaactctccggtccaccatggagtacctctcagctctgaaccccagtgacttactcaggtcagtatctaatataagctcggagtttggacggagggtctggacctcagctccaccaccccagcgacctttccgtgtctgtgatcacaagcggaccatccggaaaggcctgacagctgccacccgccaggagctgctagccaaagcattggagaccctactgctgaatggagtgctaaccctggtgctagaggaggatggaactgcagtggacagtgaggacttcttccagctgctggaggatgacacgtgcctgatggtgttgcagtctggtcagagctggagccctacaaggagtggagtgctgtcatatggcctgggacgggagaggcccaagcacagcaaggacatcgcccgattcacctttgacgtgtacaagcaaaaccctcgagacctctttggcagcctgaatgtcaaagccacattctacgggctctactctatgagttgtgactttcaaggacttggcccaaagaaagtactcagggagctccttcgttggacctccacactgctgcaaggcctgggccatatgttgctgggaatttcctccacccttcgtcatgcagtggagggggctgagcagtggcagcagaagggccgcctccattcctactaaggggctctgagcttctgcccccagaatcattccaaccgacccactgcaaagactatgacagcatcaaatttcaggacctgcagacagtacaggctagataacccacccaatttccccactgtcctctgatcccctcgtgacagaacctttcagcataacgcctcacatcccaagtctatacccttacctgaagaatgctgttctttcctagccacctttctggcctcccacttgccctgaaaggccaagatcaagatgtcccccaggcatcttgatcccagcctgactgctgctacatctaatcccctaccaatgcctcctgtccctaaactccccagcatactgatgacagccctctctgactttaccttgagatctgtcttcatacccttcccctcaaactaacaaaaacatttccaataaaaatatcaaatatttaccactaa

SEQ ID NO: 2 - RNA, 19 nt, synthetic construct
  source 1..19: mol_type = other RNA
cttactcaggtcagtatct

SEQ ID NO: 3 - RNA, 19 nt, synthetic construct
  source 1..19: mol_type = other RNA
atgtcaaagccacattcta

SEQ ID NO: 4 - RNA, 19 nt, synthetic construct
  source 1..19: mol_type = other RNA
ctatgagttgtgactttca

SEQ ID NO: 5 - RNA, 19 nt, synthetic construct
  source 1..19: mol_type = other RNA
tatgagttgtgactttcaa

SEQ ID NO: 6 - RNA, 19 nt, synthetic construct
  source 1..19: mol_type = other RNA
gactatgacagcatcaaat

SEQ ID NO: 7 - RNA, 19 nt, synthetic construct
  source 1..19: mol_type = other RNA
ctatgacagcatcaaattt

SEQ ID NO: 8 - RNA, 19 nt, synthetic construct
  source 1..19: mol_type = other RNA
ctgaagaatgctgttcttt

SEQ ID NO: 9 - RNA, 19 nt, synthetic construct
  source 1..19: mol_type = other RNA
cctgaaaggccaagatcaa

SEQ ID NO: 10 - RNA, 19 nt, synthetic construct
  source 1..19: mol_type = other RNA
cttgagatctgtcttcata

SEQ ID NO: 11 - RNA, 19 nt, synthetic construct
  source 1..19: mol_type = other RNA
ggcccaaagaaagtactca

SEQ ID NO: 12 - RNA, 19 nt, synthetic construct
  source 1..19: mol_type = other RNA
caggtcagtatctaatata

SEQ ID NO: 13 - RNA, 19 nt, synthetic construct
  source 1..19: mol_type = other RNA
gactgctgctacatctaat

SEQ ID NO: 14 - RNA, 19 nt, synthetic construct
  source 1..19: mol_type = other RNA
gactatgacagcatcaaat

SEQ ID NO: 15 - RNA, 19 nt, synthetic construct
  source 1..19: mol_type = other RNA
cccactgcaaagactatga

SEQ ID NO: 16 - RNA, 19 nt, synthetic construct
  source 1..19: mol_type = other RNA
gctctactctatgagttgt

SEQ ID NO: 17 - RNA, 19 nt, synthetic construct
  source 1..19: mol_type = other RNA
gccatatgttgctgggaat

SEQ ID NO: 18 - RNA, 19 nt, synthetic construct
  source 1..19: mol_type = other RNA
gacttactcaggtcagtat

SEQ ID NO: 19 - RNA, 19 nt, synthetic construct
  source 1..19: mol_type = other RNA
ccaaagcattggagaccct

SEQ ID NO: 20 - RNA, 19 nt, synthetic construct
  source 1..19: mol_type = other RNA
aatataagctcggagtttg

SEQ ID NO: 21 - RNA, 19 nt, synthetic construct
  source 1..19: mol_type = other RNA
ccgcctccattcctactaa

SEQ ID NO: 22 - RNA, 19 nt, synthetic construct
  source 1..19: mol_type = other RNA
gaggatgacacgtgcctga

SEQ ID NO: 23 - RNA, 19 nt, synthetic construct
  source 1..19: mol_type = other RNA
aggtcagtatctaatataa

SEQ ID NO: 24 - RNA, 19 nt, synthetic construct
  source 1..19: mol_type = other RNA
ggaatttcctccacccttc

SEQ ID NO: 25 - RNA, 19 nt, synthetic construct
  source 1..19: mol_type = other RNA
gattcacctttgacgtgta

SEQ ID NO: 26 - RNA, 19 nt, synthetic construct
  source 1..19: mol_type = other RNA
tcaggtcagtatctaatat

SEQ ID NO: 27 - RNA, 19 nt, synthetic construct
  source 1..19: mol_type = other RNA
ctctatgagttgtgacttt

SEQ ID NO: 28 - RNA, 19 nt, synthetic construct
  source 1..19: mol_type = other RNA
agaggaggatggaactgca

SEQ ID NO: 29 - RNA, 19 nt, synthetic construct
  source 1..19: mol_type = other RNA
gattcacctttgacgtgta

SEQ ID NO: 30 - RNA, 19 nt, synthetic construct
  source 1..19: mol_type = other RNA
gccacattctacgggctct

SEQ ID NO: 31 - RNA, 19 nt, synthetic construct
  source 1..19: mol_type = other RNA
gaaagtactcagggagctc

SEQ ID NO: 32 - RNA, 19 nt, synthetic construct
  source 1..19: mol_type = other RNA
gtctatacccttacctgaa

SEQ ID NO: 33 - RNA, 19 nt, synthetic construct
  source 1..19: mol_type = other RNA
gctgctagccaaagcattg

SEQ ID NO: 34 - RNA, 19 nt, synthetic construct
  source 1..19: mol_type = other RNA
ggagtggagtgctgtcata

SEQ ID NO: 35 - RNA, 19 nt, synthetic construct
  source 1..19: mol_type = other RNA
ggccaagatcaagatgtcc

SEQ ID NO: 36 - RNA, 19 nt, synthetic construct
  source 1..19: mol_type = other RNA
cttactcaggtcagtatct

SEQ ID NO: 37 - RNA, 19 nt, synthetic construct
  source 1..19: mol_type = other RNA
ctatgagttgtgactttca

SEQ ID NO: 38 - RNA, 19 nt, synthetic construct
  source 1..19: mol_type = other RNA
gactatgacagcatcaaat

SEQ ID NO: 39 - RNA, 19 nt, synthetic construct
  source 1..19: mol_type = other RNA
ctatgacagcatcaaattt

SEQ ID NO: 40 - RNA, 19 nt, synthetic construct
  source 1..19: mol_type = other RNA
ctgaagaatgctgttcttt

SEQ ID NO: 41 - RNA, 19 nt, synthetic construct
  source 1..19: mol_type = other RNA
cctgaaaggccaagatcaa

SEQ ID NO: 42 - RNA, 19 nt, synthetic construct
  source 1..19: mol_type = other RNA
cttgagatctgtcttcata

SEQ ID NO: 43 - RNA, 19 nt, synthetic construct
  source 1..19: mol_type = other RNA
cctcaaactaacaaaaaca

SEQ ID NO: 44 - RNA, 19 nt, synthetic construct
  source 1..19: mol_type = other RNA
ctcaaactaacaaaaacat

SEQ ID NO: 45 - RNA, 19 nt, synthetic construct
  source 1..19: mol_type = other RNA
caaactaacaaaaacattt

SEQ ID NO: 46 - RNA, 19 nt, synthetic construct
  source 1..19: mol_type = other RNA
ctaacaaaaacatttccaa

SEQ ID NO: 47 - RNA, 19 nt, synthetic construct
  source 1..19: mol_type = other RNA
caaaaacatttccaataaa

SEQ ID NO: 48 - RNA, 19 nt, synthetic construct
  source 1..19: mol_type = other RNA
ccaataaaaatatcaaata

SEQ ID NO: 49 - RNA, 19 nt, synthetic construct
  source 1..19: mol_type = other RNA
caataaaaatatcaaatat

SEQ ID NO: 50 - RNA, 19 nt, synthetic construct
  source 1..19: mol_type = other RNA
agatactgacctgagtaag

SEQ ID NO: 51 - RNA, 19 nt, synthetic construct
  source 1..19: mol_type = other RNA
tagaatgtggctttgacat

SEQ ID NO: 52 - RNA, 19 nt, synthetic construct
  source 1..19: mol_type = other RNA
tgaaagtcacaactcatag

SEQ ID NO: 53 - RNA, 19 nt, synthetic construct
  source 1..19: mol_type = other RNA
ttgaaagtcacaactcata

SEQ ID NO: 54 - RNA, 19 nt, synthetic construct
  source 1..19: mol_type = other RNA
atttgatgctgtcatagtc

SEQ ID NO: 55 - RNA, 19 nt, synthetic construct
  source 1..19: mol_type = other RNA
aaatttgatgctgtcatag

SEQ ID NO: 56 - RNA, 19 nt, synthetic construct
  source 1..19: mol_type = other RNA
aaagaacagcattcttcag

SEQ ID NO: 57 - RNA, 19 nt, synthetic construct
  source 1..19: mol_type = other RNA
ttgatcttggcctttcagg

SEQ ID NO: 58 - RNA, 19 nt, synthetic construct
  source 1..19: mol_type = other RNA
tatgaagacagatctcaag

SEQ ID NO: 59 - RNA, 19 nt, synthetic construct
  source 1..19: mol_type = other RNA
tgagtactttctttgggcc

SEQ ID NO: 60 - RNA, 19 nt, synthetic construct
  source 1..19: mol_type = other RNA
tatattagatactgacctg

SEQ ID NO: 61 - RNA, 19 nt, synthetic construct
  source 1..19: mol_type = other RNA
attagatgtagcagcagtc

SEQ ID NO: 62 - RNA, 19 nt, synthetic construct
  source 1..19: mol_type = other RNA
atttgatgctgtcatagtc

SEQ ID NO: 63 - RNA, 19 nt, synthetic construct
  source 1..19: mol_type = other RNA
tcatagtctttgcagtggg

SEQ ID NO: 64 - RNA, 19 nt, synthetic construct
  source 1..19: mol_type = other RNA
acaactcatagagtagagc

SEQ ID NO: 65 - RNA, 19 nt, synthetic construct
  source 1..19: mol_type = other RNA
attcccagcaacatatggc

SEQ ID NO: 66 - RNA, 19 nt, synthetic construct
  source 1..19: mol_type = other RNA
atactgacctgagtaagtc

SEQ ID NO: 67 - RNA, 19 nt, synthetic construct
  source 1..19: mol_type = other RNA
agggtctccaatgctttgg

SEQ ID NO: 68 - RNA, 19 nt, synthetic construct
  source 1..19: mol_type = other RNA
caaactccgagcttatatt

SEQ ID NO: 69 - RNA, 19 nt, synthetic construct
  source 1..19: mol_type = other RNA
ttagtaggaatggaggcgg

SEQ ID NO: 70 - RNA, 19 nt, synthetic construct
  source 1..19: mol_type = other RNA
tcaggcacgtgtcatcctc

SEQ ID NO: 71 - RNA, 19 nt, synthetic construct
  source 1..19: mol_type = other RNA
ttatattagatactgacct

SEQ ID NO: 72 - RNA, 19 nt, synthetic construct
  source 1..19: mol_type = other RNA
gaagggtggaggaaattcc

SEQ ID NO: 73 - RNA, 19 nt, synthetic construct
  source 1..19: mol_type = other RNA
tacacgtcaaaggtgaatc

SEQ ID NO: 74 - RNA, 19 nt, synthetic construct
  source 1..19: mol_type = other RNA
gattcacctttgacgtgta

SEQ ID NO: 75 - RNA, 19 nt, synthetic construct
  source 1..19: mol_type = other RNA
aaagtcacaactcatagag

SEQ ID NO: 76 - RNA, 19 nt, synthetic construct
  source 1..19: mol_type = other RNA
tgcagttccatcctcctct

SEQ ID NO: 77 - RNA, 19 nt, synthetic construct
  source 1..19: mol_type = other RNA
tacacgtcaaaggtgaatc

SEQ ID NO: 78 - RNA, 19 nt, synthetic construct
  source 1..19: mol_type = other RNA
agagcccgtagaatgtggc

SEQ ID NO: 79 - RNA, 19 nt, synthetic construct
  source 1..19: mol_type = other RNA
gagctccctgagtactttc

SEQ ID NO: 80 - RNA, 19 nt, synthetic construct
  source 1..19: mol_type = other RNA
ttcaggtaagggtatagac

SEQ ID NO: 81 - RNA, 19 nt, synthetic construct
  source 1..19: mol_type = other RNA
caatgctttggctagcagc

SEQ ID NO: 82 - RNA, 19 nt, synthetic construct
  source 1..19: mol_type = other RNA
tatgacagcactccactcc

SEQ ID NO: 83 - RNA, 19 nt, synthetic construct
  source 1..19: mol_type = other RNA
ggacatcttgatcttggcc

SEQ ID NO: 84 - RNA, 19 nt, synthetic construct
  source 1..19: mol_type = other RNA
agatactgacctgagtaag

SEQ ID NO: 85 - RNA, 19 nt, synthetic construct
  source 1..19: mol_type = other RNA
tgaaagtcacaactcatag

SEQ ID NO: 86 - RNA, 19 nt, synthetic construct
  source 1..19: mol_type = other RNA
atttgatgctgtcatagtc

SEQ ID NO: 87 - RNA, 19 nt, synthetic construct
  source 1..19: mol_type = other RNA
aaatttgatgctgtcatag

SEQ ID NO: 88 - RNA, 19 nt, synthetic construct
  source 1..19: mol_type = other RNA
aaagaacagcattcttcag

SEQ ID NO: 89 - RNA, 19 nt, synthetic construct
  source 1..19: mol_type = other RNA
ttgatcttggcctttcagg

SEQ ID NO: 90 - RNA, 19 nt, synthetic construct
  source 1..19: mol_type = other RNA
tatgaagacagatctcaag

SEQ ID NO: 91 - RNA, 19 nt, synthetic construct
  source 1..19: mol_type = other RNA
tgtttttgttagtttgagg

SEQ ID NO: 92 - RNA, 19 nt, synthetic construct
  source 1..19: mol_type = other RNA
atgtttttgttagtttgag

SEQ ID NO: 93 - RNA, 19 nt, synthetic construct
  source 1..19: mol_type = other RNA
aaatgtttttgttagtttg

SEQ ID NO: 94 - RNA, 19 nt, synthetic construct
  source 1..19: mol_type = other RNA
ttggaaatgtttttgttag

SEQ ID NO: 95 - RNA, 19 nt, synthetic construct
  source 1..19: mol_type = other RNA
tttattggaaatgtttttg

SEQ ID NO: 96 - RNA, 19 nt, synthetic construct
  source 1..19: mol_type = other RNA
tatttgatatttttattgg

SEQ ID NO: 97 - RNA, 19 nt, synthetic construct
  source 1..19: mol_type = other RNA
atatttgatatttttattg